(19) United States
(12) Patent Application Publication
(10) Pub. No.: US 2023/0084263 A1
(43) Pub. Date: Mar. 16, 2023

(54) SWEETENING INGREDIENTS

(71) Applicants: LUCOZADE RIBENA SUNTORY LIMITED, Uxbridge Greater London (GB); BRAIN BIOTECH AG, Zwingenberg (DE)

(72) Inventors: Alice CANCELLIERI, Greater London (GB); Zafer GEZGIN, Greater London (GB); Paul Stephen JONES, Coleford Gloucestershire (GB); Caroline Jane SAUNDERS, Uxbridge Greater London (GB); Roberto VANIN, Uxbridge Greater London (GB); Juergen ECK, Zwingenberg (DE); Guido MEURER, Zwingenberg (DE); Claudia BORGMEIER, Zwingenberg (DE)

(21) Appl. No.: 17/799,865

(22) PCT Filed: Feb. 15, 2021
(86) PCT No.: PCT/EP2021/053670

§ 371 Date: Aug. 15, 2022

(30) Foreign Application Priority Data
Feb. 14, 2020 (GB) 2002078.0

(51) Int. Cl. A23L 2/60; A23L 33/135; A23L 33/14; A23L 33/105

(52) U.S. Cl. CPC A23L 2/60; A23L 33/135; A23L 33/14; A23L 33/105

(57) ABSTRACT

The present invention relates to ingredients, particularly sweetening ingredients, for example ingredients for use in reduced sugar, low-sugar, or zero-sugar beverage or food products. More specifically, the present invention relates to ingredients derived from plant infusions, in particular fermented infusions of stevia.

### FIELD OF THE INVENTION

The present invention relates to ingredients, particularly sweetening ingredients, for example ingredients for use in reduced sugar, low-sugar, or zero-sugar beverage or food products. More specifically, the present invention relates to ingredients derived from plant infusions, in particular infusions of stevia (e.g. Stevia rebaudiana).

### BACKGROUND

The South American plant, Stevia rebaudiana, is known for its sweet-tasting leaves. The sweet taste comes from naturally occurring compounds within the stevia plant, called steviol glycosides. However, stevia can also taste bitter, have liquorice-like aromas, and sometimes metallic and astringent mouthfeels. These other sensory attributes of stevia are undesirable and limit its use as a sweetening ingredient in its natural form.

Sweetening ingredients and additives derived from, or based on, stevia, have previously been developed. A problem with many sweeteners derived from stevia is that they can still tend to have a liquorice aftertaste, which is undesirable to many consumers in terms of their taste in soft drinks. Another problem associated with stevia-derived sweeteners is that their taste can linger on the tongue for longer than the sugar temporal profile that consumers are accustomed to. This means that the application and market size for using these sweeteners is considerably limited.

The general approach, to address the limitations of the natural taste of stevia, has been to extract, isolate and/or concentrate the compounds responsible for the sweet taste, i.e. the steviol glycosides. These compounds are the main ingredients (or precursors) of many sweeteners marketed under the generic name ‘stevia’ and several trade names. More than 50 steviol glycosides have been found in S. rebaudiana leaves, including stevioside, steviolbioside, dulcoside A and many rebaudiosides. Steviol glycosides from Stevia rebaudiana have been reported to be between 30 and 350 times sweeter than sucrose. They are heat-stable, pH-stable, and are often described as being non-fermentable (see, e.g., Journal of Medicinal Plants Research; Vol. 7(46), pp. 3343-3353, 10 Dec. 2013). Additionally, they do not induce a glycemic response when ingested, because humans cannot metabolize steviol glycosides, which makes them attractive as natural sugar substitutes for diabetics and other people on carbohydrate-controlled diets (see, e.g., S.M. Savita et al (2004), Journal of Human Ecology, Vol. 15 (4), pp. 261-264). Steviol glycosides and related sweet molecules (e.g. diterpenic and triterpenic sweet molecules, such as the suaviosides and mogrosides) also occur in related species such as Stevia phlebophylla, in the plants Rubus chingii (Rosacae) and Rubus suavissimus (Chinese blackberry), and in the fruit of the gourd vine, luo han guo (Siraitia grosvenorii) or monk fruit.

In typical stevia leaves, up to over 95% of the steviol glycosides contain 1-4 glucose units. Steviol glycosides of higher glycosylation (>4 glucose units) have an improved taste but are present only at very low concentrations and so are significantly more expensive. Solvent extraction, currently used to isolate these more highly glycosylated glycosides, is not ideal given the large volumes of solvent being used and necessary number of purification steps. A number of companies utilise enzymes to glycosylate stevia extracts. However, enzymatically glycosylated steviol glycosides are not approved as sweeteners in the EU, and obtaining regulatory approval for use in food is not straightforward. Another approach has been to use recombinant (i.e. genetically modified) microorganisms such as bacteria and yeasts to produce desirable steviol glycoside compounds. However, many consumers would prefer to avoid products which involve the use of genetically modified organisms, so this is also not ideal.

There is therefore a need for alternative natural sweetening ingredients based on stevia, with superior taste credentials, wider consumer appeal and greater utility. The present invention has been devised in light of these considerations.

### SUMMARY OF THE INVENTION

The present inventors have devised a novel and inventive approach to this problem, which focusses on stevia itself as a natural plant ingredient and uses natural processes to modify and improve its flavour and hence increase its utility as a sweetener. This is in direct contrast to the majority of approaches currently being pursued elsewhere, which are based on extracting or isolating particular chemical compounds from the stevia plant, or on producing those compounds synthetically.

The present invention accordingly provides a natural ingredient based on a fermented infusion of stevia. In some aspects the invention provides a natural ingredient comprising a fermented infusion of stevia.

The ingredient of the invention has a taste and sensory profile which is modified (improved) as a result of the fermentation (i.e. when compared to an unfermented stevia infusion). For example, undesirable flavour compounds, including those responsible for bitter liquorice notes and/or woody notes, and/or green, grassy, ‘tea-like’ flavours, may be reduced or absent. Furthermore, in some embodiments, certain flavour compounds may be present, enriched, or enhanced in the fermented infusion, which contribute to the improved taste.

In one aspect, the invention provides an ingredient, i.e. a sweetening ingredient, e.g. for a foodstuff or a beverage, comprising a fermented infusion of stevia, wherein the infusion has been fermented using a microorganism which is preferably yeast or bacteria or a combination thereof.

In these aspects, the yeast and/or bacteria may be selected, e.g. via a screening process, so as to produce a pre-determined sensory and/or taste profile in the final (fermented) product.

In another aspect, the invention provides an ingredient based on a fermented infusion of stevia, wherein the fermented infusion is obtainable by contacting stevia (e.g. fresh or dried stevia leaves) with water and heating to produce an infusion, then directly contacting a fermentation microorganism with said infusion (e.g. by adding the fermentation microorganism directly to said infusion).

In another aspect, the invention provides an ingredient, i.e. a sweetening ingredient, e.g. for a foodstuff or a beverage, comprising a fermented infusion of stevia, wherein the fermented infusion is prepared by contacting stevia (e.g. fresh or dried stevia leaves) with water and optionally heating to produce an infusion, then directly contacting a fermentation microorganism with said infusion (e.g. by adding the fermentation microorganism directly to said infusion).

In another aspect, the present invention provides an ingredient based on a fermented infusion of stevia, wherein the infusion has been fermented using a combination of at least two different microorganisms. In some embodiments, the infusion has been fermented using a combination of at least one yeast and at least one bacteria.

A further aspect of the present invention is an ingredient, for example a sweetening ingredient, comprising steviol glycosides in aqueous solution and having physicochemical properties, for example a pH, optical density, lactate and acetate content, as described herein.

In a further aspect, also provided is a solid ingredient, e.g. a sweetening ingredient, obtained by or obtainable by drying an ingredient as described herein. In some embodiments, the solid ingredient may be formulated, for example, as a granulated sweetener or a sweetening tablet.

In one aspect, the present invention provides a process for preparing an ingredient, i.e. a sweetening ingredient, comprising contacting stevia (e.g. fresh or dried stevia leaves) with water and heating to produce an infusion, then contacting a fermentation microorganism directly with said infusion (e.g. by adding the fermentation microorganism directly to said infusion).

For example, the process may comprise the steps of:

(a) contacting stevia (e.g. cut, native or dried stevia leaf) with water, preferably with heating to a temperature of 40-90° C., or at a temperature of 40-80° C., or at a temperature of 50-70° C., to produce an infusion;

(b) (optionally) adding a carbohydrate feedstock, preferably a sugar, to the infusion;

(c) (optionally) filtering the infusion to remove remaining stevia;

(d) contacting the infusion with a fermentation microorganism, e.g. by adding the fermentation microorganism to the infusion;

(e) fermenting the infusion under conditions suitable to the microorganism; and

(f) (optionally) filtering the fermented infusion to remove the microorganism.

Also provided by the present invention is an ingredient obtainable using a process as described herein. Also provided is an ingredient obtained using a process as described herein.

In some embodiments of the products and processes of the invention, the microorganism used to ferment the infusion is, or comprises, a yeast, for example a yeast of the family Saccharomycetaceae. In some embodiments the microorganism used for the fermentation is a yeast selected from: Saccharomyces cerevisiae, Kluyveromyces lactis, Kluyveromyces marxianus, Zygosaccharomyces Pichia membranifaciens, Cyberlindnera jadinii, and Meyerozyma guilliermondii.

In some embodiments products and processes of the invention, the microorganism used to ferment the infusion is, or comprises a bacterium, for example a lactic-acid producing bacterium.

In some embodiments, more than one microorganism is used for the fermentation. For example, a combination of two or more yeasts, a combination of two or more bacteria, or a combination of one or more yeasts with one or more bacteria. In these embodiments, fermentation with more than one microorganism may take place sequentially or simultaneously.

In some embodiments, the microorganism used for the fermentation comprises a combination of one or more yeasts with one or more bacteria, wherein the yeast is preferably selected from Kluyveromyces lactis, Kluyveromyces marxianus, Zygosaccharomyces rouxii, Pichia membranifaciens, Cyberlindnera jadinii, and Meyerozyma guilliermondii, and wherein the bacterium is preferably of the Lactobacillus genus, and is more preferably selected from Lactobacillus delbrueckii, Lactobacillus fructivorans, and Lactobacillus acidophilus.

In a further aspect, the present invention provides the use of a sweetening ingredient as described herein in the production of a food or beverage product. Also provided is a food or beverage product, preferably a reduced sugar, low-sugar or sugar-free food or beverage product, comprising a sweetening ingredient as described herein.

The present invention expressly encompasses any combination of the aspects and preferred features described herein, except where such a combination is clearly impermissible or expressly avoided.

### SUMMARY OF THE FIGURES

Embodiments and experiments illustrating the principles of the invention will now be discussed with reference to the accompanying figures:

FIG. 1. Sensory profile of fermented stevia infusion prepared in accordance with the invention (see Example 2; stevia infusions of varying strength fermented for 2 days using S. cerevisiae yeast). Results are compared to the unfermented stevia infusion. Modalities assessed include appearance (Ap), Aroma (Ar), Flavour (F), Aftertaste (At). FIG 1a shows profile for 1.2 g/L stevia (unfermented vs. fermented); FIG. 1 b shows profile for 5 g/L stevia (unfermented vs. fermented); FIG. 1c shows profile for 10 g/L stevia (unfermented vs. fermented).

FIG. 2. A, Base Peak Chromatogram (BPC) “fermented sample” (grey) vs “unfermented control”(black) (Example 7, Table 1, sample 10, HPLC method I) with intensity on y-axis over time on x-axis; B, MS-spectrum (intensity on the y-axis over m/z on x-axis) at the elution time of the peak of interest (marked with arrow); B1, MS-spectrum peak (RT 13 min); B2, MS-spectrum peak (RT 22.5 min).

FIG. 3. A, Base Peak Chromatogram (BPC) “fermented sample”(grey) vs “unfermented control”(black) (Example 7, Table 1, sample 07, HPLC method I) with intensity on y-axis over time on x-axis; B, MS-spectrum (intensity on the y-axis over m/z on x-axis) at the elution time of the peak of interest (marked with arrow); B1, MS-spectrum peak (RT 13 min); B2, MS-spectrum peak (RT 22.5 min).

FIG. 4. A, Base Peak Chromatogram (BPC) “fermented sample” (grey) vs “unfermented control” (black) (Example 7, Table 2, sample 05_06, HPLC method I) with intensity on y-axis over time on x-axis; B, MS-spectrum (intensity on the y-axis over m/z on x-axis) at the elution time of the peak of interest (marked with arrow); B1, MS-spectrum peak (RT 13 min); B2, MS-spectrum peak (RT 22.5 min).

FIG. 5. A, Base Peak Chromatogram (BPC) “fermented sample” (grey) vs “unfermented control” (black) (Example 7, Table 2, sample 07_08, HPLC method I) with intensity on y-axis over time on x-axis; B, MS-spectrum (intensity on the y-axis over m/z on x-axis) at the elution time of the peak of interest (marked with arrow); B1, MS-spectrum peak (RT 13 min); B2, MS-spectrum peak (RT 22.5 min). FIG. 6. A, Base Peak Chromatogram (BPC) “fermented sample” (grey) vs “unfermented control” (black) (Example 7, Table 1, sample 19, HPLC method I) with intensity on y-axis over time on x-axis; B, MS-spectrum (intensity on the y-axis over m/z on x-axis) at the elution time of the peak of interest (marked with arrow); B1, MS-spectrum peak (RT 13 min); B2, MS-spectrum peak (RT 22.5 min).

FIG. 7. Full sensory profiles (trained panel) of fermented stevia infusions prepared in accordance with the invention. See Example 7; Table 1; Samples 10 (dashed line) and 19 (dotted line). Results are compared to a non-fermented reference sample (Ref; solid line). Modalities assessed include appearance (Ap), Aroma (Ar), Flavour (F), Mouthfeel (Mf), Aftertaste (At). Boxed attributes show statistically significant differences at 95% confidence.

FIG. 8. A, Base Peak Chromatogram (BPC) “fermented sample” (grey) vs “unfermented control” (black) (Example 7B, sample S015B; HPLC method II) with intensity on y-axis over time on x-axis; B, MS-spectrum (intensity on the y-axis over m/z on x-axis) at the elution time of the peak of rubusoside standard (marked with arrow).

FIG. 9. Sensory results from shortbread tasting. Results for attributes appearance, overall flavour, sweetness, bitterness, overall texture, crispiness and lingering aftertaste (AT) are shown in a spider diagram. Shortbreads A (full sugar), B (half sugar+fermented stevia infusion of the invention), C (half sugar +unfermented stevia infusion), and D (half sugar+Reb A) were compared.

FIG. 10. Exemplary stevia infusion step, using continuous flow column. Stevia (8 kg) is covered with hot water (80° C.) in the column (initial volume 65 L). Further water is added continuously during an initial soaking step (˜15 mins) and a subsequent elution step where the valve at the base of the column is opened to run off the infusion (over a further ˜35 minutes). Total volume of hot water used =130 L. Total infusion time =50 mins. Final infusion volume collected =80 L.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Described herein is an ingredient, for example a sweetening ingredient based on, or comprising, a fermented infusion of stevia.

The term ‘stevia’ as used herein refers primarily to plant material from stevia (i.e. Stevia rebaudiana). Alternative plant materials may include those from related plants including, but not limited to, Stevia phlebophylla, Rubus chingii (Rosacae), Rubus suavissimus (Chinese blackberry), and monk fruit (Siraitia grosvenorii). Unless otherwise specified, the term ‘stevia’ as used herein expressly includes these related plants (i.e. plants comprising diterpenic and triterpenic sweet compounds, such as steviol glycosides, mogrosides and suavosides). Plant material includes, without limitation, the leaf, bark, vine, stem, seed, bean, nut, sap, oil, milk, bud, fruit, berry, root and/or flower. In preferred embodiments, the plant material comprises leaf. In some embodiments, the plant material may comprise waste plant material, for example pomace, which includes skins, pulp, seeds or stems or waste leaves. The plant material may comprise fresh plant material (e.g. fresh leaves) or dried plant material (e.g. dried leaves). It may be cut or chopped if desired, or used whole (e.g. whole leaves). Preparations of stevia plant material suitable for use in the present invention (for example, dried stevia leaves) are readily available e.g. from commercial sources.

In some embodiments, the stevia used in the processes and products of the invention comprises stevia leaves.

In some embodiments, the stevia used in the processes and products of the invention comprises dried stevia (e.g. dried stevia leaves). Dried stevia is stevia plant material (e.g. leaves) from which water has been removed, for example using methods known in the art (e.g. air drying, convective drying, freeze drying). Dried leaves, as used herein can be distinguished from cured leaves, which are treated under specific conditions (a curing process), which may remove water but which also chemically modify the plant material itself. In some embodiments of the present invention the stevia plant material, as supplied, is not chemically processed (e.g. by curing) before use.

In some embodiments, the stevia used in the processes and products of the invention comprises uncured stevia leaves. In some embodiments, the stevia used in the processes and products of the invention comprises dried, uncured stevia leaves.

In some embodiments, the stevia used in the processes and products of the invention comprises cut stevia e.g. cut stevia leaves. Preferably, cutting is carried out using a blade or knife (rather than, for example, a mesh or grinder). Preferably the leaves are not cut too finely. Without wishing to be bound by theory, in some embodiments it may be preferable to avoid excessively fine particles of stevia (e.g. dust or powder). Preferably, the stevia used in the processes and products of the invention is not ground, powdered or pulverised.

In some embodiments, the stevia used in the processes and products of the invention comprises cut stevia leaves. In some embodiments, the stevia used in the processes and products of the invention comprises cut, dried stevia leaves. In some embodiments, the stevia used in the processes and products of the invention comprises cut, uncured stevia leaves. In some embodiments, the stevia used in the processes and products of the invention comprises cut, dried, uncured stevia leaves. In some embodiments, the stevia leaves are cut to a size (i.e. a median diameter) of between about 1 mm and about 10 mm.

The ingredient of the present invention is based on an infusion of stevia. The term ‘infusion’ is commonly used, e.g. in the beverage industry, to refer to a drink made by soaking tea leaves, herbs, etc. in liquid, preferably water. More generally, and as used herein, the term ‘infusion’ refers to a liquid composition obtained by contacting plant material (i.e. stevia plant material as described herein) with water, preferably at an elevated temperature.

Preferably, the infusion is produced at a temperature below boiling point (i.e. below 100° C.) such that organic compounds from the plant material (e.g. the flavour and aroma compounds including, but not limited to, steviol glycosides) are gently dissolved into the water. This can be distinguished from methods used in the prior art to produce ‘extracts’ of stevia, wherein the plant material is e.g. boiled vigorously in water and/or other solvent(s), sometimes repeatedly i.e. over multiple extraction steps, and is often then further concentrated e.g. in vacuo, to maximise the yield of organic compounds removed from the plant. The infusions used in the present invention are distinct from these highly concentrated ‘extracts’ of stevia.

Methods and processes for producing infusions of stevia for use in the present invention are further described below.

To produce an ingredient, e.g. a sweetening ingredient, according to the present invention, the infusion of stevia may be subject to a fermentation step. Fermentation can be generally defined as a metabolic process in which a microorganism (e.g. a yeast, a fungus or bacteria; either active cells or resting cells) converts carbohydrate (i.e. starch or sugar) into alcohol or acids and/or carbon dioxide. Fermentative modification of other organic compounds present in the substrate (fermentation medium) occurs concurrently, resulting in further changes to the chemical composition of the substrate. The term ‘fermented’ as used in the context of food and beverage products has been defined by the Food and Agriculture Organisation of the United Nations (see: http://www.fao.org/biotech/C11doc.htm) as the process of bioconversion of organic substances by microorganisms and/or enzymes (complex proteins) of microbial, plant or animal origin. The term ‘fermented’ as used herein may be construed accordingly. In particular embodiments however it may refer, more specifically, to a product which has been subjected to a fermentation process by inoculation with a suitable microorganism, preferably in the presence of a suitable carbohydrate feedstock.

Fermentation methods and processes, suitable for use in the present invention, are further described below.

The ingredient of the invention has a taste and sensory profile which is modified (improved) as a result of the fermentation process (i.e. when compared to an unfermented stevia infusion). Surprisingly and advantageously, the present inventors have found that an improved taste and sensory profile can be obtained by subjecting an infusion of stevia (e.g. an infusion of stevia leaf) to a natural fermentation process i.e. by adding a fermentation microorganism such as yeast or bacteria to the infusion and then fermenting under appropriate conditions. For example, undesirable flavour compounds, including those responsible for bitter liquorice notes and/or woody notes and/or green, grassy, ‘tea-like’ flavours may be reduced or eliminated from the fermented infusion. In some embodiments, the fermented infusion has reduced bitter liquorice flavours; in some embodiments, the fermented infusion has reduced woody flavours; and/or in some embodiments the fermented infusion has reduced green/grassy flavours; when compared to an unfermented stevia infusion.

In some embodiments the amount of certain volatile compounds including, but not limited to, terpenoids such as alpha-pinene, beta-bourbonene, alpha-bergamotene, and spathulenol, may be decreased in the infusion after fermentation.

In some embodiments the amount of certain volatile compounds including, but not limited to ethanol, 2-methyl-1-propanol, 3-methylbutanal, 2-methylbutanol, 3-methylbutyric acid, 2-methylbutyric acid, 3-methylbutyl acetate, 2-methylbutyl acetate, butoxyacetic acid, benzaldehyde, ethyl hexanoate, benzenacetaldehyde, alpha-dimethylstyrene, benzeneethanol, octanoic acid, ethyl octanoate, nonanoic acid, decanoic acid, beta-damascenone, 9-decenoic acid, and ethyl decanoate may be increased in the infusion after fermentation.

Furthermore, in some embodiments, certain flavour compounds, which contribute to the improved taste, may be present in the fermented infusion and/or may be enhanced or increased in the fermented infusion. In other words, fermentation of the infusion may shift or alter the composition of the stevia infusion, when compared to the composition before fermentation. Without wishing to be bound by theory, it is thought that, in some embodiments, the relative proportions of particular steviol glycosides and/or related compounds, having flavour-enhancing properties, may be increased by fermentation. In some embodiments, surprisingly, the relative proportions of steviol glycoside compounds are substantially unchanged, but the sensory profile is nevertheless significantly modified and/or improved. Without wishing to be bound by theory this is thought to be primarily as a result of other changes in the composition resulting from the inventive fermentation process.

In some embodiments, changes in the composition of in the fermented infusion are indicated by the presence of novel markers in a spectroscopic analysis, for example in an LC-MS spectrum. For example, the present inventors have noted that, in some embodiments of the present invention, new peaks having m/z 1127 and m/z 701 are detected in the fermented infusion, which are not found (i.e. are below detectable limits) in an unfermented stevia infusion (mass spectrometric detection using a Bruker AmazonSL lonTrap in negative mode, scan range 500-1200 m/z). The peaks are detected at RT 13 min and 23 min respectively (LC-MS using a Phenomenex Synergi column: 2.5 μ Hydro-RP 100 A, 100*2; Solvent A: 0.04% acetic acid; Solvent B: methanol +0.04% acetic acid; Flow: isocratic 50%B with 0.25 ml/min).

Without wishing to be bound by theory, it is thought that these peaks may represent steviol glycosides or related compounds which are produced, enriched, or enhanced by the fermentation reaction. In some embodiments of the present invention, therefore, the sweetening ingredient described herein comprises a fermented infusion of stevia comprising at least one steviol glycoside compound with a molecular weight of about 1128 (corresponding to m/z 1127 in negative mode) which was not detected in the unfermented infusion. In some embodiments, the sweetening ingredient comprises a fermented infusion of stevia comprising at least one steviol glycoside compound with a molecular weight of about 702 (corresponding to m/z 701 in negative mode) which was not detected in the unfermented infusion.

The present inventors have also found that in some embodiments, the relative proportion of certain steviol glycoside compounds, in particular of Rubusoside, may be increased by the fermentation process. Without wishing to be bound by theory, since Rubusoside has fewer sugar (glycoside) units compared to the other steviol glycosides such as RebA and RebG it is possible that, in these embodiments, some of these steviol glycosides have been converted to Rubusoside during the fermentation process.

In some embodiments, the weight ratio of Rubusoside to the sum of Rebaudioside A, Rebaudioside B, Rebaudioside C, Rebaudioside D, Stevioside, Rebaudioside F, Rebaudioside M, Rebaudioside N, Dulcoside A, Rebaudioside I, Rebaudioside G, Rubusoside, Steviobioside and Rebaudioside E in the sweetening ingredient of the invention is from about 0.5% to about 15%, about 1.0% to about 15%, about 1.5% to about 15%, about 2.0% to about 15%, about 2.5% to about 15%, about 3.0% to about 15%, about 3.5% to about 15%, about 4.0% to about 15%, about 4.5% to about 15%, about 5.0% to about 15%, about 5.5% to about 15%, about 6.0% to about 15%, about 6.5% to about 15%, about 0.5% to about 14%, about 0.5% to about 13%, about 0.5% to about 12%, about 0.5% to about 11%, about 0.5% to about 10%, about 0.5% to about 9.5%, about 0.5% to about 9.0%, about 0.5% to about 8.5%, about 0.5% to about 8.0%, about 1.0% to about 14%, about 1.5% to about 13%, about 2.0% to about 12%, about 2.5% to about 11%, about 3.0% to about 10%, about 3.5% to about 9.5%, or about 4.0% to about 9.0%.

In some embodiments, the mole ratio of Rubusoside to sum of Rebaudioside A, Rebaudioside B, Rebaudioside C, Rebaudioside D, Stevioside, Rebaudioside F, Rebaudioside M, Rebaudioside N, Dulcoside A, Rebaudioside I, Rebaudioside G, Rubusoside, Steviobioside and Rebaudioside E in the sweetening ingredient of the invention is from about 0.5% to about 15%, about 1.0% to about 15%, about 1.5% to about 15%, about 2.0% to about 15%, about 2.5% to about 15%, about 3.0% to about 15%, about 3.5% to about 15%, about 4.0% to about 15%, about 4.5% to about 15%, about 5.0% to about 15%, about 5.5% to about 15%, about 6.0% to about 15%, about 6.5% to about 15%, about 0.5% to about 14%, about 0.5% to about 13%, about 0.5% to about 12%, about 0.5% to about 11%, about 0.5% to about 10%, about 1.0% to about 14%, about 1.5% to about 13%, about 2.0% to about 12%, about 2.5% to about 11%, or about 3.0% to about 10%.

Advantageously, the present inventors have found that, by appropriate selection of fermentation microorganism(s) and optimisation of process conditions, it is possible to produce a fermented infusion matching a target profile. In some embodiments, the target profile comprises a pre-determined sensory and/or taste profile. In some embodiments, the target profile comprises (additionally or alternatively) pre-determined analytical criteria. Analytical criteria may include, for example, the presence or absence of certain compounds in the composition, or a particular ratio of certain components, such as particular steviol glycoside compounds, which may be assessed by spectroscopic methods. More broadly, analytical criteria may include, for example, the presence or absence of certain spectroscopic markers, e.g. the presence or absence of certain peaks in an LC-MS spectrum.

In some embodiments, analytical criteria indicative of a target sensory profile may include the pH of the fermented infusion, as further described below. In some embodiments, analytical criteria indicative of a target sensory profile may include the optical density of the fermented infusion, as further described below. In some embodiments, analytical criteria indicative of a target sensory profile may include the content or concentration of one or more metabolites including, but not limited to, lactate and acetate, as further described below.

The sweetening ingredients described herein comprise a fermented infusion of stevia, wherein the fermented infusion is preferably obtainable by, or obtained by: contacting stevia (e.g. dried stevia leaves) with water and heating, then contacting/adding a fermentation microorganism directly with/to said infusion.

Processes for preparing the sweetening ingredients of the present invention are generally described herein. For example, the process may comprise the steps of:

(a) contacting stevia with water, and preferably heating, to produce an infusion;

(b) (optionally) adding a carbohydrate (e.g. a sugar) or a carbohydrate source to the infusion;

(c) (optionally) filtering the infusion to remove remaining stevia;

(d) contacting the infusion with a fermentation microorganism (e.g. adding said fermentation microorganism to the infusion); (e) fermenting the infusion under conditions suitable to the microorganism; and (f) (optionally) filtering the fermented infusion.

The labels (a) to (f) above are not to be regarded as limiting. As would be understood by the person skilled in the art, the steps may be carried out in any technically reasonable order. It will also be understood that despite being written as separate ‘steps’, in some embodiments certain actions may be performed simultaneously. For example, as explained further below, in some embodiments, carbohydrate may be added to the infusion along with the fermentation microorganism (e.g. pre-activated yeast).

The infusion step (i.e. step (a) in the above example) comprises contacting (i.e. mixing, combining) the stevia plant material with water and, preferably, heating.

In some embodiments, the infusion step comprises heating to a temperature above about 40° C. In some embodiments, the infusion step comprises heating to a temperature above about 50° C. In some embodiments, the infusion step comprises heating to a temperature above about 60° C.

In some embodiments, the infusion step comprises heating to a temperature below about 100° C. In some embodiments, the infusion step comprises heating to a temperature below about 90° C. In some embodiments, the infusion is heated to a temperature below about 85° C. In some embodiments, the infusion is heated to a temperature below about 80° C. In some embodiments, the infusion is heated to a temperature below about 70° C.

In some embodiments the temperature is about 40-90° C. In some embodiments the temperature is about 40-85° C. In some embodiments the temperature is about 50-90° C. In some embodiments the temperature is about 40-90° C. In some embodiments the temperature is about 50-85° C. In some embodiments the temperature is about 40-80° C. In some embodiments the temperature is about 50-80° C. In some embodiments the temperature is about 40-70° C. In some embodiments the temperature is about 50-70° C. In some embodiments the temperature is about 60° C. In some embodiments the temperature is about 70° C. In some embodiments the temperature is about 80° C.

In some embodiments, the duration of the infusion step — i.e. the length of time during which the stevia plant material is in contact with the (hot) water: the ‘steep’ time—is less than about 120 minutes. In some embodiments, the duration of the infusion step is less than about 90 minutes. In some embodiments, the duration of the infusion step is less than about 75 minutes. In some embodiments, the duration of the infusion step is less than about 60 minutes. In some embodiments, the duration of the infusion step is less than about 45 minutes. In some embodiments, the duration of the infusion step is less than about 30 minutes.

In some embodiments, the duration of the infusion step is longer than about 10 minutes. In some embodiments, the duration of the infusion step is longer than about 15 minutes. In some embodiments, the duration of the infusion step is longer than about 20 minutes.

In some embodiments, the duration of the infusion step is from 10 to 75 minutes. In some embodiments, the duration of the infusion step is from 15 to 60 minutes. In some embodiments, the duration of the infusion step is from 30 to 60 minutes. In some embodiments, the duration of the infusion step is from 15 to 45 minutes. In some embodiments, the duration of the infusion step is from 30 to 45 minutes. In some embodiments, the duration of the infusion step is about 30 minutes.ln some embodiments, the infusion is produced by mixing stevia with water at a concentration (w/v) greater than about 15 g/L. In some embodiments, the infusion is produced by mixing stevia with water at a concentration (w/v) greater than about 20 g/L. In some embodiments, the infusion is produced by mixing stevia with water at a concentration (w/v) greater than about 30 g/L. In some embodiments, the infusion is produced by mixing stevia with water at a concentration (w/v) greater than about 60 g/L.

In some embodiments, the infusion is produced by mixing stevia with water at a concentration (w/v) lower than about 180 g/L. In some embodiments, the infusion is produced by mixing stevia with water at a concentration (w/v) lower than about 150 g/L. In some embodiments, the infusion is produced by mixing stevia with water at a concentration (w/v) lower than about 120 g/L. In some embodiments, the infusion is produced by mixing stevia with water at a concentration (w/v) lower than about 100 g/L. In some embodiments, the infusion is produced by mixing stevia with water at a concentration (w/v) lower than about 90 g/L.

In some embodiments, the infusion is produced by mixing stevia with water at a concentration (w/v) between about 15 g/L and about 150 g/L. In some embodiments, the infusion is produced by mixing stevia with water at a concentration (w/v) between about 15 g/L and about 100 g/L. In some embodiments, the infusion is produced by mixing stevia with water at a concentration (w/v) between about 15 g/L and about 90 g/L. In some embodiments, the infusion is produced by mixing stevia with water at a concentration (w/v) between about 20 g/L and about 100 g/L. In some embodiments, the infusion is produced by mixing stevia with water at a concentration (w/v) between about 20 g/L and about 90 g/L. In some embodiments, the infusion is produced by mixing stevia with water at a concentration (w/v) between about 20 g/L and about 60 g/L. In some embodiments, the infusion is produced by mixing stevia with water at a concentration (w/v) between about 20 g/L and about 50 g/L. In some embodiments, the infusion is produced by mixing stevia with water at a concentration (w/v) between about 20 g/L and about 50 g/L.

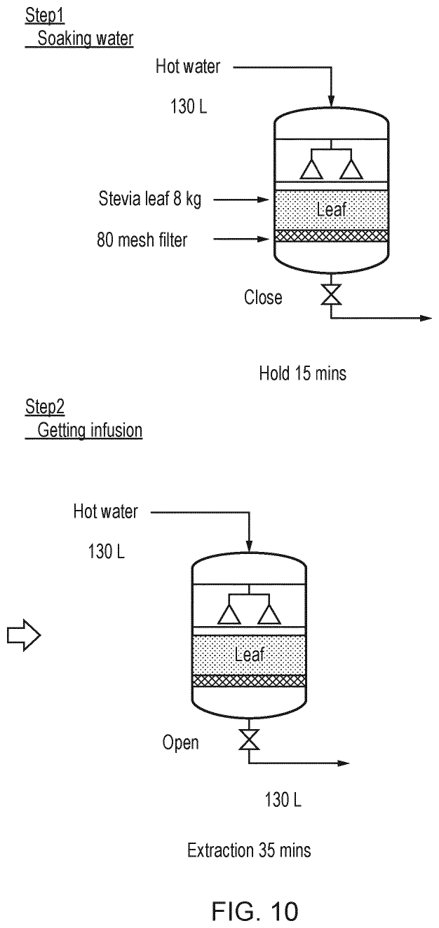

In some embodiments, the infusion may be produced in a continuous column process (see FIG. 10) with an initial volume of water (optionally heated as described above) being added to cover the stevia for an initial soaking step and the remaining volume of water being added during a second extraction step wherein the infusion is collected from the bottom of the column. In such embodiments the concentration (w/v) as set out above may be calculated relative to the total volume of water used.

Additional water may be added to the infusion prior to the fermentation step (for example, along with the microorganism or in a separate step, e.g. to top-up evaporated water after infusion or to dilute the infusion to a desired concentration). The concentrations above refer to the amount of stevia plant material present during the ‘steep’ (infusion process).

In some embodiments, at least one carbohydrate is preferably added to the stevia infusion, to be used by the microorganism(s) as a carbon source during the fermentation reaction. The carbohydrate preferably comprises a sugar, for example: glucose, sucrose, fructose, lactose, or any combination thereof. Other carbohydrates include, but are not limited to, starch, cellulose, hemicelluloses, pectin, inulin, pullulan and saccharose. In some embodiments, a carbohydrate is not added but a source of carbohydrate may be added to produce a feedstock for the fermentation in situ. For example, fibre may be converted to sugars by an added enzyme, such as a cellulase.

The carbohydrate or carbohydrate source may conveniently be added to the water along with the stevia plant material, before or during the infusion process. Some or all of the carbohydrate may also be added to the microorganism, prior to its addition to the infusion. For example, in particular when the microorganism is a yeast, some sugar may be used to ‘activate’ the yeast, prior to its addition to the infusion.

The total amount of carbohydrate added to the infusion (i.e. the amount of carbohydrate present at the start of the fermentation step) is preferably more than about 2 g/L. In some embodiments, the total amount of carbohydrate added to the infusion (i.e. the amount of carbohydrate present at the start of the fermentation step) is preferably more than about 4 g/L. In some embodiments, the total amount of carbohydrate added to the infusion (i.e. the amount of carbohydrate present at the start of the fermentation step) is preferably more than about 5 g/L. In some embodiments, the total amount of carbohydrate added to the infusion is more than about 10 g/L. In some embodiments, the total amount of carbohydrate added to the infusion is more than about 15 g/L. In some embodiments, the total amount of carbohydrate added to the infusion is more than about 20 g/L. In some embodiments, the total amount of carbohydrate added to the infusion is more than about 25 g/L. In some embodiments, the total amount of carbohydrate added to the infusion is equal to or more than about 30 g/L.

The total amount of carbohydrate added to the infusion (i.e. the amount of carbohydrate present at the start of the fermentation step) is preferably less than about 60 g/L. In some embodiments, the total amount of carbohydrate added to the infusion is less than about 50 g/L. In some embodiments, the total amount of carbohydrate added to the infusion is less than about 40 g/L. In some embodiments, the total amount of carbohydrate added to the infusion is less than about 35 g/L.

In some embodiments, the total amount of carbohydrate added to the infusion is from 2 g/L to 50 g/L.

In some embodiments, the total amount of carbohydrate added to the infusion is from 2 g/L to 35 g/L.

In some embodiments, the total amount of carbohydrate added to the infusion is from 5 g/L to 50 g/L.

In some embodiments, the total amount of carbohydrate added to the infusion is from 10 g/L to 40 g/L. In some embodiments, the total amount of carbohydrate added to the infusion is from 20 g/L to 50 g/L. In some embodiments, the total amount of carbohydrate added to the infusion is from 20 g/L to 40 g/L. In some embodiments, the total amount of carbohydrate added to the infusion is from 30 g/L to 50 g/L. In some embodiments, the total amount of carbohydrate added to the infusion is about 30 g/L.

In preferred embodiments, the carbohydrate feedstock used is a sugar. In some embodiments, the sugar is selected from glucose and sucrose. In some embodiments, the sugar is sucrose.

The total amount of sugar added to the infusion (i.e. the amount of sugar present at the start of the fermentation step) is preferably more than about 5 g/L (0.5 Bx). In some embodiments, the total amount of sugar added to the infusion is more than about 10 g/L (1 Bx). In some embodiments, the total amount of sugar added to the infusion is more than about 15 g/L (1.5 Bx). In some embodiments, the total amount of sugar added to the infusion is more than about 20 g/L (2 Bx). In some embodiments, the total amount of sugar added to the infusion is more than about 25 g/L (2.5 Bx). In some embodiments, the total amount of sugar added to the infusion is equal to or more than about 30 g/L (3 Bx).

The total amount of sugar added to the infusion (i.e. the amount of sugar present at the start of the fermentation step) is preferably less than about 60 g/L (6 Bx). In some embodiments, the total amount of sugar added to the infusion is less than about 50 g/L (5 Bx). In some embodiments, the total amount of sugar added to the infusion is less than about 40 g/L (413x). In some embodiments, the total amount of sugar added to the infusion is less than about 35 g/L (3.5 Bx).

In some embodiments, the total amount of sugar added to the infusion is from 5 g/L (0.5 Bx) to 50 g/L (5 Bx). In some embodiments, the total amount of sugar added to the infusion is from 10 g/L (1 Bx) to 40 g/L (4 Bx). In some embodiments, the total amount of sugar added to the infusion is from 20 g/L (2 Bx) to 50 g/L (5 Bx). In some embodiments, the total amount of sugar added to the infusion is from 20 g/L (2 Bx) to 40 g/L (4 Bx). In some embodiments, the total amount of sugar added to the infusion is from 30 g/L (3 Bx) to 50 g/L (5 Bx). In some embodiments, the total amount of sugar added to the infusion is about 30 g/L (3 Bx).

In some embodiments, the infusion is filtered to remove the stevia plant material (i.e. the stevia leaves), before the fermentation microorganism is added. In alternative embodiments, the plant material may be removed by other known methods such as e.g. centrifugation or decantation, References herein to filtration are intended to encompass also these methods, where appropriate. In other embodiments, the fermentation microorganism is added directly to the infusion without filtering: in some embodiments the plant material may then be removed at a later stage, along with other unwanted solids (e.g. biomass from the fermentation microorganism).

Processes for producing ingredients according to the present invention may comprise a step wherein a stevia infusion, produced as described above, is fermented using a microorganism. To start the fermentation process, a suitable microorganism (or preparation thereof) is added to the stevia infusion.

Preferably, the microorganism is added directly to the liquid product from the infusion step (after filtration and/or cooling, if applicable). Preferably, the stevia infusion prepared as set out above is used directly in the fermentation step which follows, except that in some embodiments the infusion may be filtered to remove stevia plant material and/or in some embodiments it may be diluted by the addition of further liquid (water) and/or in some embodiments it may be cooled (e.g. for temporary storage) and/or heated to an appropriate fermentation temperature. In particular, in preferred embodiments, the stevia infusion is not subjected to any chemical modification, solvent extraction, or concentration steps, prior to fermentation.

In some embodiments, additional water may be added, either separately or along with the microorganism (e.g. as part of a preparation of the microorganism). In some embodiments, the microorganism is added in a liquid preparation comprising additional water. In some embodiments, the microorganism is added in a liquid preparation comprising water and some or all of the sugar required for the fermentation.

In some embodiments of the invention, the microorganism is, or comprises, a fungus selected from: Aspergillus spp.; Ustilago spp.; or a combination thereof. In some of the embodiments, the microorganism used for fermentation comprises one or more fungi selected from: Aspergillus oryzae, Ustilago maydis; or a combination thereof.

In some embodiments of the invention, the microorganism used to ferment the infusion is, or comprises, a yeast, for example a yeast of the family Saccharomycetaceae.

In some embodiments of the invention, the microorganism is, or comprises, a yeast selected from: Saccharomyces spp.; Pichia spp.; Zygosaccharomyces spp.; Kluyveromyces spp.; Kloeckera spp.; Brettanomyces spp.; Metschnikowia spp.; Aureobasidium spp.; Issatchenkia spp.; Torulaspora spp.; Lachancea spp.; Hanseniaspora spp.; Cyberlindnera spp.; and Meyerozyma spp.or a combination thereof. In some embodiments, the microorganism is a yeast selected from Saccharomyces spp.; Kluyveromyces spp.; Zygosaccharomyces spp.; Pichia spp.; Cyberlindnera spp.; and Meyerozyma spp.; or a combination thereof.

In some embodiments, the microorganism is, or comprises, a yeast selected from: S. cerevisiae; S. uvarum; S. bayanus; S. exiguus; S. carlsbergensis; T. delbrueckii; Lachancea thermotolerans; P. anomala; P. kluyveri; P. caribbica; P. guilliermondii; Z. bailii; K. marxianus; K. lactis; M. pulcherrima; A. pullulans, I. orientalis; K. apiculata; K. javanica; H. uvarum; H. osmophilia; or a combination thereof.

In some embodiments, the microorganism used for the fermentation comprises one or more yeasts selected from: Saccharomyces cerevisiae, Kluyveromyces lactis, Kluyveromyces marxianus, Zygosaccharomyces rouxii, Pichia membranifaciens, Cyberlindnera jadinii, and Meyerozyma guilliermondii. In some embodiments, the microorganism used for the fermentation comprises one or more yeasts selected from: Zygosaccharomyces rouxii, Cyberlindnera jadinii, and Meyerozyma guilliermondii. In some embodiments, the microorganism used for the fermentation is, or comprises, Meyerozyma guilliermondii.

In some embodiments of the invention, the microorganism used to ferment the infusion is, or comprises, a bacterium, for example a lactic-acid producing bacterium.

In some embodiments of the invention, the microorganism used for the fermentation is, or comprises, bacteria, for example a lactic-acid producing bacteria, for example selected from the genera Lactobacillus (for example L. acidophilus or L. fructivorans), Leuconostoc, Pediococcus, Lactococcus (for example L. raffinolactis), Streptococcus, Aerococcus, Carnobacterium, Enterococcus, Oenococcus, Sporolacto bacillus, Tetragenococcus, Vagococcus, or Weissella. In other embodiments, the bacteria used for fermentation is selected from Zymomonas spp., preferably Z. mobilis; or Bacillus spp, for example B. stearothermophilus or B. licheniformis.

In some embodiments of the invention, the microorganism used for the fermentation is, or comprises, bacteria from the genus Lactobacillus. In some embodiments, the microorganism used for the fermentation is, or comprises, bacteria selected from L. acidophilus, L. fructivorans, L. gasseri, L. jensenii, L. delbrueckii, L. delbrueckii subsp. Bulgaricus, L. amylovorus, L, crispatus, and L. helveticus. In some embodiments, the microorganism used for the fermentation is, or comprises, Lactobacillus acidophilus, Lactobacillus fructivorans, and Lactobacillus delbrueckii. In some embodiments, the microorganism used for the fermentation is, or comprises, Lactobacillus acidophilus.

In some embodiments, more than one microorganism is used for the fermentation. For example, a combination of two or more yeasts; a combination of two or more bacteria; a combination of two or more fungi; a combination of one or more fungi with one or more bacteria; a combination of one or more yeasts with one or more fungi; or a combination of one or more yeasts with one or more bacteria.

In particular embodiments, the microorganism used for the fermentation comprises a combination of one or more yeasts with one or more bacteria, wherein the yeast is preferably selected from Kluyveromyces lactis, Kluyveromyces marxianus, Zygosaccharomyces rouxii, Pichia membranifaciens, Cyberlindnera jadinii, and Meyerozyma guilliermondii, and wherein the bacteria is preferably of the Lactobacillus genus, and is more preferably selected from Lactobacillus delbrueckii, Lactobacillus fructivorans, and Lactobacillus acidophilus.

In some embodiments, the microorganism used for the fermentation comprises a combination of a yeast selected from Saccharomyces cerevisiae, Kluyveromyces lactis, Kluyveromyces marxianus, Zygosaccharomyces rouxii, Pichia membranifaciens, Cyberlindnera jadinii, and Meyerozyma guilliermondii, with at least one bacteria, preferably a lactic-acid producing bacterium as set out above.

In some embodiments, the microorganism used for the fermentation comprises a combination of a yeast and at least one lactic-acid producing bacteria, for example a bacteria selected from L. acidophilus, L. fructivorans, L. gasseri, L. jensenii, L. delbrueckii, L. delbrueckii subsp. Bulgaricus, L. amylovorus, L, crispatus, and L. helveticus, preferably selected from Lactobacillus acidophilus, Lactobacillus fructivorans, and Lactobacillus delbrueckii.

In these embodiments, fermentation with more than one microorganism may take place separately, sequentially or simultaneously.

In some embodiments, fermentation with more than one microorganism takes place simultaneously. For example, in some embodiments two or more yeasts are added, together, to the stevia infusion and fermentation by each occurs concurrently. In other embodiments, one or more yeasts and one or more bacteria are added together to the stevia infusion (dual inoculum) and fermentation by each occurs concurrently.

In some embodiments, fermentation with more than one microorganism takes place sequentially. For example, fermentation may be carried out first with one or more yeasts and, subsequently, further fermentation may be carried out with one or more bacteria. Alternatively, fermentation may be carried out first with one or more bacteria and, subsequently, further fermentation may be carried out with one or more yeasts.

An advantageous feature of the present invention is that, depending on the choice of microorganism(s) and on the process conditions used, a variety of different ingredients, e.g. sweetening ingredients, having different properties (including but not limited to sensory properties such as taste, appearance, aroma and mouthfeel) are accessible from the stevia plant material.

Microorganisms suitable for use in the present invention are generally described herein. In some embodiments of the invention, a suitable microorganism or combination of microorganisms for use in the fermentation process may be selected via a screening process. Such a screening process may assist in identifying species and/or strains of microorganisms which are capable of producing particular desired endpoints. For example, screening may be used to identify microorganisms which are capable of producing particular flavours and/or of removing particular flavours which are present in unfermented stevia. Alternatively or additionally screening may be used to identify microorganisms which are capable of producing, enriching or enhancing particular compounds (e.g. particular steviol glycosides and/or particular volatiles) in the fermentation reaction and/or of removing (i.e. degrading or chemically modifying) other compounds which are present in unfermented stevia.

Accordingly, in some embodiments of the present invention, the microorganism used in the fermentation step is selected so as to match a particular target profile in the final product. In some embodiments of the present invention, the microorganism used in the fermentation step is selected so as to produce a pre-determined sensory and/or taste profile in the final product. In some embodiments of the present invention, the microorganism used in the fermentation step is selected so as to produce a pre-determined analytical/chemical profile in the final product.

Such a screening process may, for example, comprise performing one or more test fermentation(s) on a suitable stevia infusion (e.g. by following a process such as that described herein) and performing analytical and/or sensory tests (as are well known in the art) on the resulting fermented samples, to determine whether, or to what extent, the analytical and/or sensory profile obtained corresponds to the pre-determined targets.

The fermentation step is carried out under conditions suitable for the microorganism(s) used, as is understood in the art.

In some embodiments, the fermentation step may be batch, fed-batch or continuous.

In some embodiments, the fermentation may be performed under substantially anaerobic conditions.

In some embodiments, the fermentation may be performed under substantially aerobic conditions.

In some embodiments, before addition to the stevia infusion the microorganism may be activated. In some embodiments, in particular when the microorganism is a yeast, activation comprises mixing the microorganism with water and, preferably, adding a suitable amount of sugar, in order to start the metabolic process of fermentation in the microorganism.

The resulting preparation of the microorganism (i.e. microorganism plus water, plus sugar if applicable) is added directly to the stevia infusion, to start the fermentation step of the process described herein. Any water and sugar included in this preparation contribute to the overall water and sugar content of the stevia infusion, as noted elsewhere, and hence form part of the fermentation medium.

In some embodiments, for activation purposes, sugar is added to the microorganism in a ratio from about 10:1 to about 50:1 (sugar/yeast w/w). In some embodiments, sugar is added to the microorganism in a ratio from about 15:1 to about 35:1 w/w. In some embodiments, sugar is added to the microorganism in a ratio from about 20:1 to about 30:1 w/w. In some embodiments, sugar is added in a ratio of about 25:1 w/w.

In some embodiments, the fermentation microorganism is present in an amount of at least about 0.1 g/L in the infusion (fermentation medium). In some embodiments, the fermentation microorganism is present in an amount of at least about 0.2 g/L. In some embodiments, the fermentation microorganism is present in an amount of at least about 0.3 g/L. In some embodiments, the fermentation microorganism is present in an amount of at least about 0.4 g/L. In some embodiments, the fermentation microorganism is present in an amount of about 0.4 g/L in the fermentation medium. In some embodiments, the fermentation microorganism is present in an amount of no more than about 0.6 g/L in the fermentation medium. In some embodiments, the fermentation microorganism is present in an amount of no more than about 0.8 g/L. In some embodiments, the fermentation microorganism is present in an amount of no more than about 1 g/L. In some embodiments, the fermentation microorganism is present in an amount of no more than about 2 g/L.

In some embodiments, the fermentation step is performed at a temperature of between 15° C. and 40° C. In some embodiments, the fermentation step is performed at a temperature of between 20° C. and 35° C. In some embodiments, the fermentation step is performed at a temperature of between 25° C. and 30° C. In some embodiments, the fermentation step is performed at a temperature of between 26° C. and 28° C. In some embodiments, the fermentation step is performed at a temperature below 35° C. In some embodiments, the fermentation step is performed at a temperature below 32° C. In some embodiments, the fermentation step is performed at a temperature below 30° C. In some embodiments, the fermentation step is performed at a temperature above 15° C. In some embodiments, the fermentation step is performed at a temperature above 20° C. In some embodiments, the fermentation step is performed at a temperature above 25° C.

In some embodiments, the duration of the fermentation is at least 2 hours. In some embodiments, the duration of the fermentation is at least 4 hours. In some embodiments, the duration of the fermentation is at least 24 hours. In some embodiments, the duration of the fermentation is at least 48 hours. In some embodiments, the duration of the fermentation is at least 72 hours (3 days). In some embodiments, the duration of the fermentation step may be less than 14 days. In some embodiments, the duration of the fermentation step may be less than 10 days. In some embodiments, the duration of the fermentation step may be less than 7 days. In some embodiments, the duration of the fermentation step may be less than 5 days. In some embodiments, the duration of the fermentation step may be less than 4 days.

In some embodiments, the duration of the fermentation step is about 1 day. In some embodiments, the duration of the fermentation step is about 2 days. In some embodiments, the duration of the fermentation step is about 3 days.

In some embodiments of the invention, the duration of fermentation is determined by the consumption of the carbohydrate feedstock (i.e. the sugar). This can be monitored by methods which are known in the art. The aim is to reduce the sugar level in the infusion (the fermentation medium) to zero, or as close as possible to zero.

Accordingly, in some embodiments, fermentation is continued until the added carbohydrate is completely, or almost completely, consumed. For example, the residual carbohydrate/sugar content after fermentation may be less than 25 g/L, less than 20 g/L, less than 15 g/L, less than 10 g/L, less than 5 g/L, less than 2 g/L, or less than 1 g/L. In some embodiments, at least 5 g/L sugar, at least 10 g/L sugar, preferably at least 15 g/I sugar, most preferably at least 20 g/L sugar is consumed by the microorganism during the fermentation reaction. In preferred embodiments, after fermentation, the infusion is substantially free of sugar. Accordingly, in some embodiments, the fermented stevia infusion of the invention may be used as a ‘sugar-free’ sweetening ingredient.

In some embodiments, the pH of the infusion at the start of the fermentation may be less than about 7. In some embodiments, the pH of the infusion at the start of the fermentation may be less than about 6.5. In some embodiments, the pH of the infusion at the start of the fermentation may be less than about 6. In some embodiments, the pH of the infusion at the start of the fermentation may be less than about 5.5.

In some embodiments, the pH of the infusion at the start of the fermentation may be more than about 4. In some embodiments, the pH of the infusion at the start of the fermentation may be more than about 4.5. In some embodiments, the pH of the infusion at the start of the fermentation may be more than about 5.

In some embodiments, the pH of the infusion at the start of the fermentation may be between about 5 and about 7. In some embodiments, the pH of the infusion at the start of the fermentation may be between about 5 and about 6.5. In some embodiments, the pH of the infusion at the start of the fermentation may be between about 5 and about 6.

In some embodiments, the pH of the infusion at the end of the fermentation may be more than about 2.5. In some embodiments, the pH of the infusion at the end of the fermentation may be more than about 3. In some embodiments, the pH of the infusion at the end of the fermentation may be more than about 3.1. In some embodiments, the pH of the infusion at the end of the fermentation may be more than about 3.5. In some embodiments, the pH of the infusion at the end of the fermentation may be more than about 4. In some embodiments, the pH of the infusion at the end of the fermentation may be more than about 4.5.

In some embodiments, the pH of the infusion at the end of the fermentation may be less than about 5.

In some embodiments, the pH of the infusion at the end of the fermentation may be less than about 4.5. In some embodiments, the pH of the infusion at the end of the fermentation may be less than about 4. In some embodiments, the pH of the infusion at the end of the fermentation may be less than about 3.9. In some embodiments, the pH of the infusion at the end of the fermentation may be less than about 3.8. In some embodiments, the pH of the infusion at the end of the fermentation may be less than about 3.5.

In some embodiments, the pH of the infusion at the end of the fermentation may be between about 2.5 and about 4.5. In some embodiments, the pH of the infusion at the end of the fermentation may be between about 3 and about 4.5. In some embodiments, the pH of the infusion at the end of the fermentation may be between about 3 and about 4. In some embodiments, the pH of the infusion at the end of the fermentation may be between about 3.1 and about 3.9. In some embodiments, the pH of the infusion at the end of the fermentation may be between about 3.1 and about 3.8.

Without wishing to be bound by theory, it is thought that an observed reduction in optical density during the fermentation process is also indicative of the biotransformation process. Optical density of the infusion may be measured and monitored with a UV-vis spectrophotometer, using methods known in the art. For example, in some embodiments a cell density meter such as the UltrospecTM 10 Classic (supplied by Biochrom) may be used.

In some embodiments, the optical density measured at a wavelength of 600 nm (OD₆₀₀) of the infusion at the end of the fermentation may be less than about 1. In some embodiments, the optical density (OD₆₀₀) of the infusion at the end of the fermentation may be less than about 0.9. In some embodiments, the optical density (OD₆₀₀) of the infusion at the end of the fermentation may be less than about 0.8. In some embodiments, the optical density (OD₆₀₀) of the infusion at the end of the fermentation may be less than about 0.7. In some embodiments, the optical density (OD₆₀₀) of the infusion at the end of the fermentation may be less than about 0.6.

In some embodiments, the optical density (OD₆₀₀) of the infusion at the end of the fermentation may be more than about 0.1. In some embodiments, the optical density (OD₆₀₀) of the infusion at the end of the fermentation may be more than about 0.12. In some embodiments, the optical density (OD₆₀₀) of the infusion at the end of the fermentation may be more than about 0.15. In some embodiments, the optical density (OD₆₀₀) of the infusion at the end of the fermentation may be more than about 0.2. In some embodiments, the optical density (OD₆₀₀) of the infusion at the end of the fermentation may be more than about 0.25.

In some embodiments, the optical density (OD₆₀₀) of the infusion at the end of the fermentation may be in the range of about 0.1 to about 1. In some embodiments, the optical density (OD₆₀₀) of the infusion at the end of the fermentation may be in the range of about 0.12 to about 0.9. In some embodiments, the optical density (OD₆₀₀) of the infusion at the end of the fermentation may be in the range of about 0.15 to about 0.9. In some embodiments, the optical density (OD₆₀₀) of the infusion at the end of the fermentation may be in the range of about 0.15 to about 0.8.

Without wishing to be bound by theory, in some embodiments the content of certain metabolites has been found to be indicative of a good sensory outcome in the final ingredient. In particular, in some embodiments the amounts of lactate and acetate in the infusion at the end of the fermentation may be optimised, to achieve a desired sensory outcome (e.g. a ‘clean’ tasting ingredient or beverage).

In some embodiments, the amount of lactate in the infusion at the end of the fermentation is less than about 15 g/L. In some embodiments, the amount of lactate in the infusion at the end of the fermentation is less than about 12 g/L. In some embodiments, the amount of lactate in the infusion at the end of the fermentation is less than about 10 g/L. In some embodiments, the amount of lactate in the infusion at the end of the fermentation is less than about 8 g/L.

In some embodiments, the amount of lactate in the infusion at the end of the fermentation is more than about 0.1 g/L. In some embodiments, the amount of lactate in the infusion at the end of the fermentation is more than about 0.2 g/L. In some embodiments, the amount of lactate in the infusion at the end of the fermentation is more than about 0.5 g/L. In some embodiments, the amount of lactate in the infusion at the end of the fermentation is less than about 1 g/L.

In some embodiments, the amount of lactate in the infusion at the end of the fermentation is from about 0 to about 12 g/L. In some embodiments, the amount of lactate in the infusion at the end of the fermentation is from about 0 to about 10 g/L. In some embodiments, the amount of lactate in the infusion at the end of the fermentation is from about 0.5 to about 10 g/L.

In some embodiments, the amount of acetate in the infusion at the end of the fermentation is less than about 4 g/L. In some embodiments, the amount of acetate in the infusion at the end of the fermentation is less than about 3 g/L. In some embodiments, the amount of acetate in the infusion at the end of the fermentation is less than about 2.5 g/L. In some embodiments, the amount of acetate in the infusion at the end of the fermentation is less than about 2 g/L.

In some embodiments, the amount of acetate in the infusion at the end of the fermentation is more than about 0.1 g/L. In some embodiments, the amount of acetate in the infusion at the end of the fermentation is more than about 0.2 g/L. In some embodiments, the amount of acetate in the infusion at the end of the fermentation is more than about 0.5 g/L..

In some embodiments, the amount of acetate in the infusion at the end of the fermentation is from about 0 to about 3 g/L. In some embodiments, the amount of acetate in the infusion at the end of the fermentation is from about 0 to about 2.5 g/L. In some embodiments, the amount of acetate in the infusion at the end of the fermentation is from about 0.2 to about 2.5 g/L.

Accordingly, a further aspect of the present invention is an ingredient, for example a sweetening ingredient, comprising steviol glycosides in aqueous solution and having physicochemical properties, for example a pH, optical density, lactate and acetate content, as described herein. The ingredient is obtainable using fermentation processes as described herein.

In some embodiments, the invention hence provides an ingredient, for example a sweetening ingredient, comprising steviol glycosides in aqueous solution, and having, for example:

a pH from about 3.1 to about 3.9;

an OD₆₀₀ from about 0.15 to 0.8;

a lactate content from about 0 to about 10 g/L; and

an acetate content from about 0 to about 2.5 g/L.

Preferably the ingredient comprises at least 50 ppm total steviol glycosides. In some embodiments the ingredient comprises at least 100 ppm total steviol glycosides. In some embodiments the ingredient comprises at least 200 ppm total steviol glycosides. In some embodiments the ingredient comprises at least 500 ppm total steviol glycosides.

As noted above, in some embodiments the process optionally includes a step wherein the infusion is filtered before the fermentation microorganism is added, to remove the stevia plant material.

In some embodiments, the process optionally includes a step wherein the stevia infusion is sterilised or pasteurised (for example, by heating) to reduce the risk of contamination with other microorganisms, prior to addition of the fermentation microorganism.

In some embodiments, the process optionally includes one or more steps wherein additional liquid (e.g. water) is added to the infusion.

In some embodiments, after the fermentation step, some or all of the remaining solids, including e.g. biomass from the microorganism, may be removed or reduced, to leave a fermented infusion suitable for use as a sweetening ingredient. In some embodiments, the fermented infusion is filtered (or centrifuged, etc) to remove solids.

The resulting fermented infusion is ready to use as an ingredient, e.g. sweetening ingredient, in liquid form. In some embodiments, the final product may be pasteurised or sterilised before being packaged and/or used.

In some embodiments, the ingredient of the invention is pasteurised. In some embodiments, the process of the invention comprises a pasteurisation step. In some embodiments, pasteurisation comprises heating to a temperature of at least 70° C., at least 80° C., at least 90° C., or at least 95° C.

In some embodiments, the ingredient may be concentrated and/or dried before packaging and/or use. In some embodiments, an ingredient of the invention is dried to provide a solid ingredient. Drying may be performed by methods known in the art. In some embodiments, drying comprises, for example, evaporation, optionally at reduced pressure; freeze drying; spray drying. Depending on the carriers/auxiliaries the products may also be obtained by spray granulation; melt granulation; coacervation; coagulation; extrusion; melt extrusion; emulsion processes; coating or other suitable encapsulation processes and optionally a suitable combination of said processes

In some embodiments, the resulting solid ingredient may be formulated as a granulated or powdered product e.g. a granulated or powdered sweetener. In some embodiments, the solid ingredient may be formulated in a tablet e.g. a sweetener tablet.

The ingredients described herein may be used in the production of a food or beverage product. Accordingly, a further aspect of the present invention is the use of a sweetening ingredient, as described herein, in the production of a food or beverage product. A further aspect is a food or beverage product, preferably a reduced sugar, low-sugar or sugar-free beverage product, comprising a sweetening ingredient as described herein.

In some embodiments, the food or beverage product is a beverage including, without limitation, a squash, a cordial, a juice, an infusion, a carbonated beverage or another soft drink.

In some embodiments, advantageously, a sweetening ingredient of the invention may have less of a foaming effect than previously known stevia-based sweeteners, in particular solvent-extracted stevia preparations.

In some embodiments, the food or beverage product is a foodstuff including, without limitation, a confectionery item. In some embodiments, the foodstuff is a biscuit, cake, or other baked good. In some embodiments, the foodstuff is a sweet or chocolate product. In some embodiments, the foodstuff is a chewing gum. In some embodiments, the foodstuff is selected from condiments including, but not limited to, sauces, ketchups, dressings, or table sauces. In some embodiments, the foodstuff is a cereal product, for example a breakfast cereal, or a snack (e.g. from potato, maize, peanut). In some embodiments the foodstuff is an animal product, for example a milk product (including but not limited to dairy, ice cream, cheese etc.) or an egg product. In other embodiments, the foodstuff is a vegetable or fruit product (e.g. fruit preparations, vegetable products). In yet further embodiments, the foodstuff is selected from a soya product including, but not limited to, tofu, tempeh or soya milk. In some embodiments, the foodstuff may be a spice mixture or other seasoning.

In some embodiments, the sweetening ingredient is added to the food or beverage in an amount from about 0.2% (v/v), from about 0.5%, from about 1%, from about 1.5% or from about 2%. In some embodiments, the sweetening ingredient is added to the food or beverage in an amount up to about 2.5% (v/v), up to about 3%, up to about 4%, up to about 5% or up to about 10%. In some embodiments, the sweetening ingredient is added to the food or beverage in an amount of about 0.5% (v/v). In some embodiments, the sweetening ingredient is added to the food or beverage in an amount of about 1% (v/v). In some embodiments, the sweetening ingredient is added to the food or beverage in an amount of about 1.5% (v/v).

In some embodimenbts, the ingredient is added in an amount of about 0.01 mg/L, preferably more than about 0.1 mg/L, preferably more than about 1 mg/L, based on the total preparation. In further embodiments, the preparation comprises a total quantity in the range of 0.01 to 10 000 mg/L, 0.1 to 1000 mg/L, preferably 0.1 to 500 mg/L, particularly preferably 0.1 to 100 mg/L, of the ingredient, based on the total weight of the preparation.

In some embodiments, the sweetening ingredient is added to the food or beverage in an amount from about 0.2% (w/w), from about 0.5%, from about 1%, from about 1.5% or from about 2%. In some embodiments, the sweetening ingredient is added to the food or beverage in an amount up to about 2.5% (w/w), up to about 3%, up to about 4%, up to about 5% or up to about 10%. In some embodiments, the sweetening ingredient is added to the food or beverage in an amount of about 0.5% (w/w). In some embodiments, the sweetening ingredient is added to the food or beverage in an amount of about 1% (w/w). In some embodiments, the sweetening ingredient is added to the food or beverage in an amount of about 1.5% (w/w).

In some embodiments, the sweetening ingredient replaces the equivalent of about 2 g/L of sugar in the beverage or food product. In some embodiments, the sweetening ingredient replaces the equivalent of up to about 3 g/L of sugar in the beverage or food product. In some embodiments, the sweetening ingredient replaces the equivalent of up to about 4 g/L of sugar in the beverage or food product. In some embodiments, the sweetening ingredient replaces the equivalent of up to about 5 g/L of sugar in the beverage or food product. In some embodiments, the sweetening ingredient replaces the equivalent of up to about 10 g/L of sugar in the beverage or food product.

In some embodiments, the sweetening ingredient replaces the equivalent of about 2 g/kg of sugar in the beverage or food product. In some embodiments, the sweetening ingredient replaces the equivalent of up to about 3 g/kg of sugar in the beverage or food product. In some embodiments, the sweetening ingredient replaces the equivalent of up to about 4 g/kgL of sugar in the beverage or food product. In some embodiments, the sweetening ingredient replaces the equivalent of up to about 5 g/kg of sugar in the beverage or food product. In some embodiments, the sweetening ingredient replaces the equivalent of up to about 10 g/kg of sugar in the beverage or food product.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

General Methods

### 1. Steviol Glycosides HPLC Analysis Method (Examples 2 to 6)

1. Introduction

The samples were analysed using a method adapted from the Jefca 2017 monograph Steviol Glycosides HPLC method. They were run on an Agilent HPLC 1100 system a gradient method was utilised with a Phenomenex Luna 5 μm C18(2), 100A, (250 mm×4.6 mm, 5 μm) column, the detector was set 210 nm. The steviol glycoside content was quantified by comparison with external standards.

2. Experimental

Reagents & Standards

Steviol glycoside standard solution Jefca mixture 0.2 mg/ml was obtained from Chromadex (part No 00010175) containing the following Steviosides: Rebaudioside A, Rebaudioside B, Rebaudioside C, Rebaudioside D, Rebaudioside F, Dulcoside A, Steviolbioside. HPLC grade deionised water and HPLC grade acetonitrile was obtained from VWR.

Mobile phase

Solvent A: Deionised Water

Solvent B: Acetonitrile

The HPLC Method employed was a gradient method the same as reported in Jefca.

HPLC Gradient Time table:

| | % | % | Flow |
|---|---|---|---|
| Time | Solvent | Solvent | Rate |
| (min) | A | B | (mL/min) |
| 0 | 85 | 15 | 0.3 |
| 40 | 70 | 30 | 0.3 |
| 60 | 55 | 45 | 0.3 |
| 70 | 55 | 45 | 0.3 |
| 70.1 | 85 | 15 | 0.3 |
| 90 | 85 | 15 | 0.3 |

Standard Preparation

The standard solution was used as supplied by Chromadex.

Instrumentation and Conditions

An Agilent 1100 HPLC System including quaternary pump, a temperature controlled column compartment set at 50° C., an autosampler and VWD absorbance detector was used for the analysis. The detector was set at 210 nm. The data acquisition was done using WATER Empower 3 software. The column used for HPLC was a reversed phase Luna 5 μm C18(2), 100A, (250 mm x 4.6 mm, 5 μm) Phenomenex.

Analysis Procedure

For the RP-HPLC method the column was flushed 30ml mobile phase (85:15 deionised water: Acetonitrile). The samples were bracketed with standard at the beginning and the end of a run for accuracy of retention times. Sample injection volume 20 μl. A calibration curve was constructed using on-column dilution method taking different volumes of the Jefca standard mixture.

| | Level 1 | Level 2 | Level 3 | Level 4 | Level 5 | |
|---|---|---|---|---|---|---|
| | Injection | Injection | Injection | Injection | Injection | |
| | volume | volume | volume | volume | volume | |
| | (1 μl) | (5 μl) | (10 μl) | (15 μl) | (20 μl) | |
| Rebaudioside D | 9.15 | 45.75 | 91.5 | 137.25 | 183 | mg/l |
| Rebaudioside A | 9.55 | 47.75 | 95.5 | 143.25 | 191 | mg/l |
| Stevioside | 9.3 | 46.5 | 93 | 139.5 | 186 | mg/l |
| Rebaudioside F | 9.45 | 47.25 | 94.5 | 141.75 | 189 | mg/l |
| Rebaudioside C | 9.25 | 46.25 | 92.5 | 138.75 | 185 | mg/l |
| Dulcoside A | 9.5 | 47.5 | 95 | 142.5 | 190 | mg/l |
| Rubusoside | 8.85 | 44.25 | 88.5 | 132.75 | 177 | mg/l |
| Rebaudioside B | 9 | 45 | 90 | 135 | 180 | mg/l |
| Steviolbioside | 9.25 | 46.25 | 92.5 | 138.75 | 185 | mg/l |

2. Volatiles Analysis HPLC Method

Extraction of volatiles by Headspace Solid Phase Micro Extraction (SPME) for GC/MS Analysis

An appropriate amount of sample was transferred to a 20 ml vial, which was then sealed. Vials were equilibrated at 75° C. for 5 minutes with agitation. The headspace of the vial was then sampled for 5 minutes at 75° C. with agitation using carboxen/polydimethylsiloxane /divinylbenzene coated SPME fibre. The volatiles adsorbed onto the fibre were analysed by thermal desorption at 270° C. in the injector port of the GC/MS.

GC/MS Analysis of Volatiles

Analysis was carried out an Agilent 7890A gas chromatograph (GC) and Agilent 7200 accurate mass Q-TOF mass spectrometer (MS) via CTC Combi-Pal autosampler.

GC/MS conditions were as follows:

Column : 30 mm x 0.25 mm fused silica with ZB-semivolatiles stationary phase

Helium carrier gas flow rate: 1 mL min⁻¹

Injector temperature: 270° C.

Column temperature: 5 min at 40° C.; then 4° C. min⁻¹ to 200° C.; then 30° C. min-1 to 350° C., hold for 3 minutes
MS analysis mode: Scam (33-350 m/z)

Peaks were tentatively identified by spectral matching with the MIST library of mass spectral data.

3. Sensory Testing—Exemplary Methodology

Sensory tests are performed using a Quantitative Descriptive Analysis (QDA). A QDA evaluates if there are differences between samples and what the differences are (qualitative=description element, and by how much the samples differ=quantitative element). This methodology is run with sensory panellists who are a group of consumers who have been screened for their sensory acuity and trained on their senses to articulate what they perceive when consuming a beverage. They have also been trained on a range of sensory methodologies. The measure obtained from a sensory panel is objective. A minimum of 6-7 panellists participate in the studies and results are taken in duplicate to ensure reproducibility.

The samples are presented first all at a time to the sensory trained panel who, during a round table discussion, initially agree on the sensory attributes or descriptors which best describe the products, in order to develop a sensory vocabulary. A sensory scientist moderates the discussion and collects the attributes selected by the panel which are clear for them, easily defined and describe differences between the samples under study.

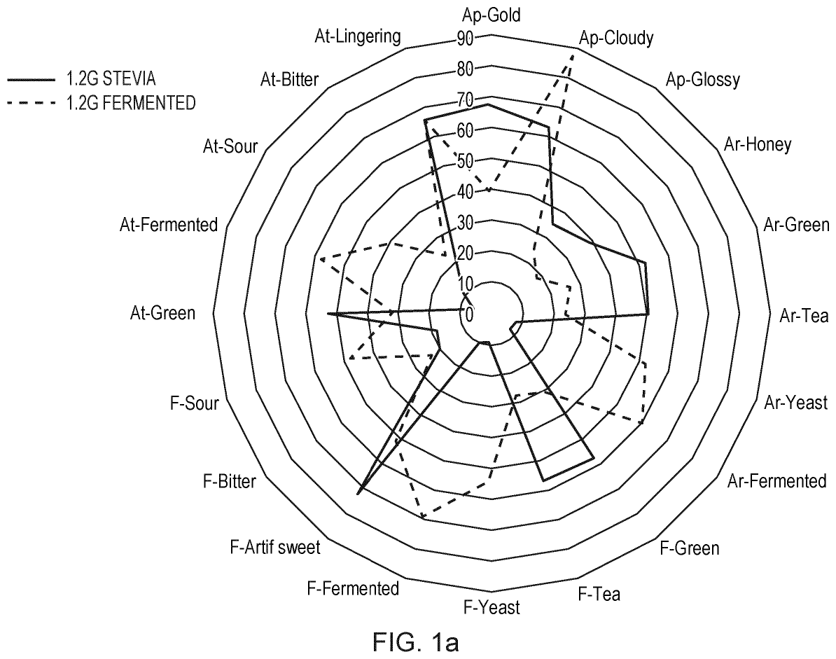

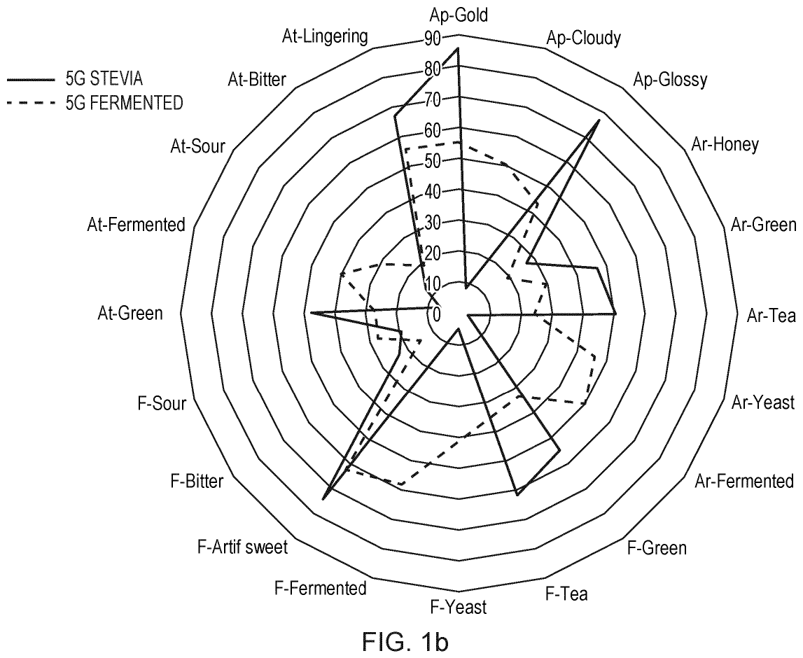

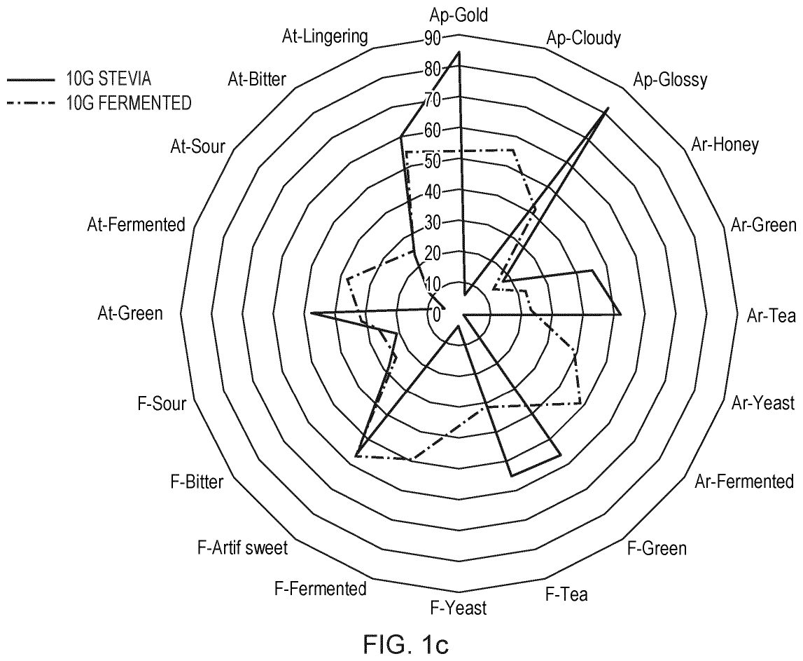

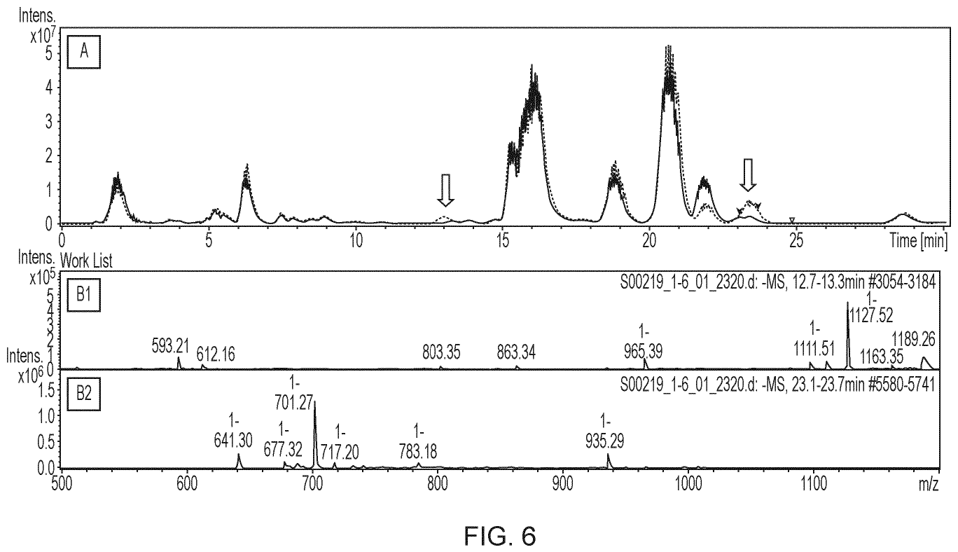

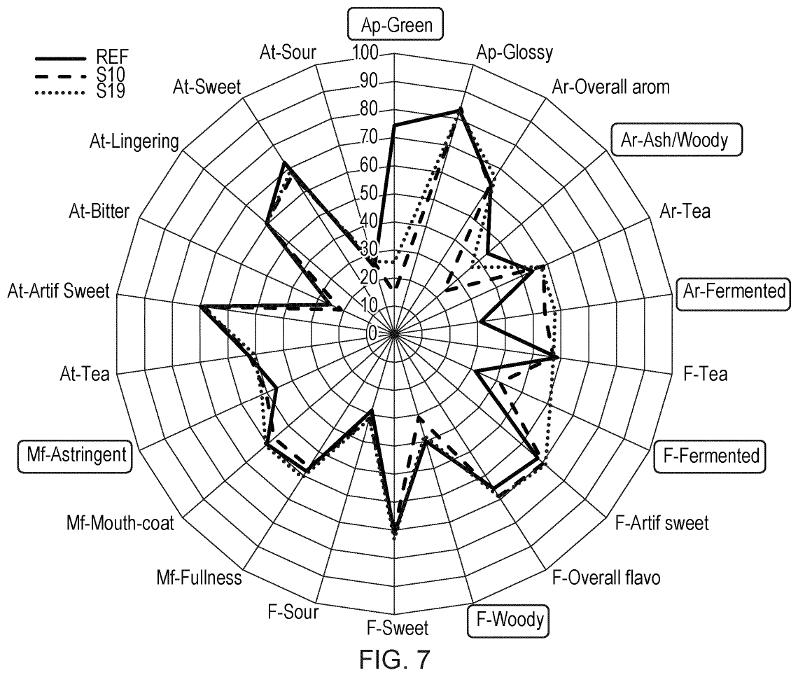

Then sensory evaluation takes place in sensory booths where each panellist assesses the samples individually. The panellists rate one sample at a time in an undefined line scale from Low to High (0-100 of each attribute). The samples are presented in a randomised and balanced order to avoid first order and carry over effects. The data collection is done online via FIZZ (Sensory Software) and the data analysis carried out with FIZZ and XLSTAT. The outcome of the data analysis is a spider graph (see e.g. FIGS. 1 and 7). Statistical Significance testing in the form of ANOVA (Analysis of Variance) and an LSD test is performed. This determines on which attributes the samples are significantly different (if any). P-value is taken as <0.05 (using 95% confidence).

### Example 1

### General Protocol for Preparation of Fermented Stevia Infusion

Stevia leaf (dried) is added to water at the desired temperature. The leaves are allowed to steep at this temperature (brewing stage) for the required infusion time and the infusion is optionally then filtered to remove the spent leaves. Further (cold) water is added if needed, to dilute the infusion to a required volume and/or reduce its temperature. A carbohydrate feedstock (e.g. sugar) may be added to the infusion (e.g. during the steep, or at any point thereafter).

Alternatively, in some embodiments, a continuous column method to prepare the stevia infusion may be used (see FIG. 10).

The microorganism (e.g. the yeast) is prepared by suspending in water and pre-activation, if necessary (e.g. by addition of sugar).

The infusion is heated or cooled if needed, to an appropriate temperature for fermentation. The preparation of the microorganism is then added to the infusion, which is allowed to ferment.

Fermentation process is monitored analytically using methods which are known in the art.

Exemplary pH-meter, densimeter and refractometer used for basic analytics:

pH Meter: Mettler Toledo Seven Easy

Refractometer: Bellingham and Stanley RFM340+

Density Meter: Anton Paar DMA 4500M

Monitoring of fermentation progress can also be assessed, for example, using spectroscopic methods such as the Acetoscan' machine manufactured by CETOTEC GmbH.

The microorganism may be removed (e.g. by filtration) after the fermentation step is complete.

An exemplary filtration comprises a plate and frame filter with cellulose filter sheets (Beco KD3 200×200 filter sheets from Eaton filtration products; rated at 10microns). For a 600 litre ferment, typically 30 sheets would be used, getting 800-1000 g of yeast that can be scraped off. The pump supplying liquid pressurises to 30psi; typical expected loss of 15-20 litres of liquid during filtration.

The resultant product is optionally pasteurised (e.g. by heating)—see Example 3.

### Example 2

### Fermentation with S. Cerevisiae Yeast

Experimental parameters:

| Jar | Raw material, |
|---|---|
| # | supplier and dose g/L |
| A | Stevia (Mane) @1.2 g/L |
| B | Stevia (Mane) @5 g/L |
| C | Stevia (Mane) @10 g/L |

| Infusion (step 1): | | |
|---|---|---|
| Hot water | 1500 | mL |
| Time (tea steep) | 45 | min |
| Temp (start) | 70 | ° C. |
| Temp (end) | 60 | ° C. |
| Fermentation (step 2): | | |
| Sugar (3Bx) | 30 | g/L |
| Yeast | 2 | g |
| Infusion (from step 1) | 1500 | mL |
| Cold Water | 2500 | mL |
| Water for Yeast | 1000 | mL |

Stevie Infusion Preparation:

Sugar: 30 g/L total sugar per jar was used, to get around 3Bx for each jar. For 5 L of ferment this corresponds to 150 g of sugar. 100 g was added and dissolved in the infusion, with the leaves. Remaining part of the sugar, 50 g, was used for the yeast activation (below).

Yeast: A strain of S. cerevisiae yeast was used, at 0.4 g/L. A total of 2 g per jar was pre-activated for all jars. The yeast was activated separately, the total of 2 g were dissolved in 1 L of water with the remaining part of the sugar (50 g) and kept there for 105 min.

Before adding the yeast to the infusion, it was checked that the solution has a temperature inferior to 30° C. to be sure not to kill the yeast.

Water: The remaining part of the 5 L of water was added before addition of the activated yeast solution to quickly bring down the temperature below 30° C.

Results:

Monitoring of Fermentation Reaction:

| | Jar | Start | Day 1 | Day 2 |
|---|---|---|---|---|
| | # | (0 h) | (18 h) | (42 h) |
| Brix | A | 3.27 | 2.88 | 2.49 |
| | B | 3.27 | 2.68 | 1.8 |
| | C | 3.34 | 2.59 | 1.52 |
| pH | A | 5.57 | 3.95 | 3.52 |
| | B | 5.57 | 3.97 | 3.5 |
| | C | 5.57 | 4.04 | 3.58 |
| Density | A | 1.01302 | 1.01075 | 1.00819 |
| (SG) | B | 1.01289 | 1.00944 | 1.00597 |
| | C | 1.01299 | 1.00847 | 1.00399 |

Steviol Glycosides Analysis:

Steviol Glycosides HPLC analysis - unfermented

| Jar #: | A d1 | B d1 | C d1 | |
|---|---|---|---|---|
| Reb D | 3.7 | 17.4 | 32.6 | mg/l |
| Reb A | 88.6 | 320 | 541.5 | mg/l |
| Stevioside | 30 | 117 | 194.6 | mg/l |
| Reb F | 2.4 | 21 | 41.5 | mg/l |
| Reb C | 11.6 | 52.2 | 103 | mg/l |
| Dulcoside A | nd | 7.3 | 26.8 | mg/l |
| Rubusoside | nd | 4.7 | 12.7 | mg/l |
| Reb B | nd | 7.6 | 16.7 | mg/l |
| Steviolbioside | nd | 6.5 | 16.3 | mg/l |
| Total steviol glycosides | 136.4 | 553.8 | 985.7 | mg/l |

Steviol Glycosides HPLC analysis - fermented

| Jar #: | A D3 | B D3 | C D3 | Units |
|---|---|---|---|---|
| Reb D | 3.9 | 15.1 | 28.1 | mg/l |
| Reb A | 90.4 | 309.4 | 572.9 | mg/l |
| Stevioside | 29.5 | 109.1 | 193 | mg/l |
| Reb F | 2.7 | 10.8 | 49.4 | mg/l |
| Reb C | 11.6 | 48.3 | 90.5 | mg/l |
| Dulcoside A | | 3.9 | 8.8 | mg/l |
| Rubusoside | | 4.5 | 9.5 | mg/l |
| Reb B | | 5.1 | 10.1 | mg/l |
| Steviolbioside | | 5 | 11 | mg/l |
| Total steviol glycosides | 138.1 | 511.2 | 973.3 | |

Sensory Profile:

As shown in FIG. 1, a significant modification in the sensory profile of the stevia infusion is achieved by fermentation.

Volatiles Analysis:

Volatiles analysis performed using analytical HPLC showed the following changes in composition.

Decrease in Terpenoid Compounds After Fermentation:

| Compound | Descriptor |
|---|---|
| alpha-Pinene | fresh, sweet, pine, earthy |
| beta-Bourbonene | herbal, woody, floral, balsamic |
| alpha-Bergamotene | woody, warm, tea |
| Spathulenol | earthy, herbal, fruity |

Increased After Fermentation:

| Compound | Descriptor |
|---|---|
| Ethanol | alcoholic |
| 2-Methyl-1-propanol | ethereal, wine |
| 3-Methylbutanal | alcoholic, fruity |
| 2-Methylbutanol | alcoholic, fruity |
| 3-Methylbutyric acid | sour, sweaty, cheesy |
| 2-Methylbutyric acid | acidic, fruity, cheesy |
| 3-Methylbutyl acetate | sweet, fruity, solvent |
| 2-Methylbutyl acetate | fruity |
| Butoxyacetic acid | |
| Benzaldehyde | almond |
| Ethyl hexanoate | fruity |
| Benzenacetaldehyde | green, sweet, floral |
| alpha-Dimethylstyrene | phenolic, plastic, clove |
| Benzeneethanol | floral |
| Octanoic acid | fatty, oily, cheesy |
| Ethyl octanoate | fruity |
| Nonanoic acid | fatty, cheesy, dairy |
| Decanoic acid | rancid, sour, fatty |
| beta-Damascenone | sweet, fruity, earthy, floral |
| 9-Decenoic acid | green, fruity, fatty, soapy |
| Ethyl decanoate | sweet, waxy, fruity |

### Example 3

### Fermentation with S. Cerevisiae Yeast; Addition of Pasteurisation Step

Experimental Parameters:

| Jar | Raw material, |
|---|---|
| # | supplier and dose g/L |
| A | Stevia (Natura) @1.2 g/L |
| B | Stevia (Natura) @5 g/L |
| C | Stevia (Natura) @10 g/L |

| Infusion (step 1): | | |
|---|---|---|
| Hot water | 1500 | mL |
| Time (tea steep) | 30 | min |
| Temp (start) | 65 | ° C. |
| Temp (end) | / | ° C. |
| Fermentation (step 2): | | |
| Sugar (3Bx) | 30 | g/L |
| Yeast | 2 | g |
| Infusion (from step 1) | 1500 | mL |
| Cold Water | 2500 | mL |
| Water for Yeast | 1000 | mL |

The stevia infusion was prepared and fermented (using a S. cerevisiae strain) using the same method as for Example 2.

Results:

Monitoring of fermentation reaction:

| | Jar | Start | Day 1 | Day 2 |
|---|---|---|---|---|
| | # | (0 h) | (18 h) | (42 h) |
| Brix | A | 3.2 | 2.85 | 2.47 |
| | B | 3.22 | 2.61 | 2.02 |
| | C | 3.26 | 2.49 | 1.82 |
| pH | A | 5.35 | 4.1 | 3.73 |
| | B | 5.21 | 4.04 | 3.66 |
| | C | 5.12 | 4.06 | 3.67 |
| Density | A | 1.01089 | 1.00844 | 1.00639 |
| (SG) | B | 1.01063 | 1.00692 | 1.00395 |
| | C | 1.0108 | 1.00581 | 1.00181 |

Steviol alycosides analysis:

Steviol Glycosides HPLC analysis - fermented

| | A | B | C | Unit |
|---|---|---|---|---|
| Reb D | 5.5 | 22.2 | 37.3 | mg/l |
| Reb A | 36.1 | 392.2 | 673.7 | mg/l |
| Stevioside | 108.0 | 132.6 | 231.8 | mg/l |
| Reb F | 3.8 | 11.3 | 22.6 | mg/l |
| Reb C | 14.8 | 52.6 | 92.8 | mg/l |
| Dulcoside A | | 3.9 | 6.7 | mg/l |
| Rubusoside | 3.4 | 8.3 | 16.2 | mg/l |
| Reb B | | 6.5 | 11.9 | mg/l |
| Steviolbioside | | 7.9 | 13.3 | mg/l |
| Total steviol glycosides | 171.6 | 637.5 | 1106.3 | mg/l |

Pasteurisation step:

The fermented infusion was pasteurised by heating using a water bath, with temperatures taken manually using a thermometer and recorded to give a pasteurisation curve as shown in the table below:

| Time | Display (° C.) | Bottle (° C.) |
|---|---|---|
| 02:32 | 46.3 | 41.8 |
| 02:38 | 52 | 46.8 |
| 3.02 | 73.2 | 68.9 |
| 3.04 | 74.9 | 70.1 |
| 3.05 | 75.3 | 70.9 |
| 3.06 | 76.4 | 71.8 |
| 3.07 | 77 | 72.6 |
| 3.08 | 76.7 | 73.3 |
| OFF | 76 | 73.7 |

Pasteurisation may also be carried out using a Miele Pasteur and a datalogger with a probe that automatically measures the temperature.

### Example 4

### Fermentation with S. Cerevisiae Yeasts; Comparison with Other Plant Ingredients

Experimental parameters:

| Jar # | Raw material, supplier and dose g/L | Yeast strain |
|---|---|---|
| C | Stevia (Natura SAS) @ 10 g/L | i |
| E | Stevia (Natura SAS) @ 10 g/L | ii |
| F | Stevia (Natura SAS) @ 30 g/L | i |
| G | Monk Fruit Concentrate (MFC) @ 2 g/L | i |
| H | Stevia (Natura) @5g/L + MFC @ 1 g/L | i |
| I | Stevia (Layn WS1905002) @ 10 g/L | i |
| J | Stevia (Layn WS1905005) @ 10 g/L | ii |
| K | Stevia (M-B) @ 10 g/L | ii |
| L | Chinese Blackberry leaves (Layn) | ii |
| | @ 10 g/L | |
| M | Chinese Blackberry leaves (M-B) @ 10 g/L | ii |

| Infusion (step 1): | | |
|---|---|---|
| Hot water | 1400 | mL |
| Time (tea steep) | 30 | min |
| Temp (start) | 65 | ° C. |
| Temp (end) | / | ° C. |
| Fermentation (step 2): | | |
| Sugar (3Bx) | 30 | g/L |
| Yeast | 1.2 | g |
| Infusion (from step 1) | 1000 | mL |
| Cold Water | 1400 | mL |
| Water for Yeast | 600 | mL |

The infusion was prepared and fermented (using S. cerevisiae yeast strains) using a method as described for Example 2.

Results:

Monitoring of fermentation reactions:

| | Jar # | Start (0 h) | Day 1 (18 h) | Day 2 (42 h) |
|---|---|---|---|---|
| Brix | C | 2.92 | 2.33 | 1.16 |
| | E | 3.08 | 2.29 | 1.79 |
| | F | 3.05 | 1.59 | 1.27 |
| | G | 4.19 | 3.92 | 3.54 |
| | H | 3.2 | 2.31 | 1.74 |
| | I | 2.44 | 1.19 | 0.62 |
| | J | 3.07 | 2.57 | 2.2 |
| | K | 3.4 | 2.49 | 1.76 |
| | L | 3.11 | 2.67 | 2.26 |
| | M | 3.16 | 2.68 | 2.27 |
| PH | C | 4.9 | 3.96 | 3.63 |
| | E | 5.48 | 4.2 | 3.89 |
| | F | 4.88 | 4.15 | 4 |
| | G | 5.2 | 3.98 | 3.57 |
| | H | 4.96 | 3.88 | 3.57 |
| | I | 5.23 | 4.14 | 3.92 |
| | J | 5.65 | 4.51 | 4.33 |
| | K | 5.85 | 4.24 | 3.98 |
| | L | 5.7 | 4.58 | 4.41 |
| | M | 5.54 | 4.45 | 4.27 |
| Density (SG) | C | 1.01038 | 1.00509 | 1.00128 |
| | E | 1.01006 | 1.00528 | 1.0018 |
| | F | 1.01095 | 1.00281 | 1.00101 |
| | G | 1.01486 | 1.01286 | 1.01066 |
| | H | 1.01138 | 1.00717 | 1.00361 |
| | I | 1.00733 | 1.00221 | 0.9988 |
| | J | 1.01026 | 1.00739 | 1.00483 |
| | K | 1.0114 | 1.00573 | 1.0002 |
| | L | 1.01045 | 1.00764 | 1.00525 |
| | M | 1.01053 | 1.00751 | 1.00538 |

Steviol glycosides analysis:

| | C | E | F | I | J | K | Unit |
|---|---|---|---|---|---|---|---|
| 1st analysis | | | | | | | |
| Reb D | 35.1 | 52.9 | 18.3 | 36.5 | nd | 3.6 | mg/l |
| Reb A | 684.3 | 642.7 | 1515.5 | 357.2 | nd | 366.0 | mg/l |
| Stevioside | 235.1 | 220.2 | 545.1 | 263.3 | nd | 258.7 | mg/l |
| Reb F | 21.6 | 20.4 | 58.5 | 13.3 | nd | 13.3 | mg/l |
| RebC | 90.1 | 84.2 | 210.4 | 58.0 | nd | 55.3 | mg/l |
| Dulcoside A | 5.8 | 5.8 | 15.0 | 7.6 | nd | 8.5 | mg/l |
| Rubusoside | 6.8 | 6.0 | 12.1 | 4.5 | 183.2 | 10.6 | mg/l |
| Reb B | 11.4 | 11.3 | 28.0 | 6.9 | nd | 4.5 | mg/l |
| Steviolbioside | 13.6 | 12.9 | 32.1 | 10.1 | nd | 7.0 | mg/l |
| Total Steviol | 1103.6 | 1056.4 | 2435.1 | 757.5 | 183.2 | 727.5 | mg/l |
| Glycosides | | | | | | | |
| 2nd analysis | | | | | | | |
| Reb D | 33.3 | 31.9 | 84.7 | 18.5 | 24.4 | 21.7 | mg/l |
| Reb A | 661.1 | 621.2 | 1465.8 | 351.8 | nd | 362.9 | mg/l |
| Stevioside | 230.1 | 215.0 | 542.8 | 259.6 | nd | 261.2 | mg/l |
| Reb F | 24.1 | 20.1 | 53.1 | 15.9 | nd | 3.8 | mg/l |
| RebC | 92.9 | 85.1 | 217.0 | 61.2 | nd | 2.9 | mg/l |
| Dulcoside A | 7.5 | 6.9 | 17.6 | 9.1 | nd | 8.8 | mg/l |
| Rubusoside | 13.3 | 16.9 | 32.9 | 18.6 | 174.7 | 22.9 | mg/l |
| Reb B | 12.2 | 11.7 | 30.7 | 7.3 | nd | 6.1 | mg/l |
| Steviolbioside | 15.7 | 14.9 | 35.3 | 11.7 | 5.5 | 8.7 | mg/l |
| Total Steviol | 1090.2 | 1023.6 | 2480.0 | 753.7 | 204.6 | 699.0 | mg/l |
| Glycosides | | | | | | | |

### Example 5

### Optimisation of Infusion Step

Experimental parameters:

| Jar # | Raw material, supplier and dose g/L |
|---|---|
| 90/60 | Stevia (Natura Greece) @ 90 g/L |
| 90/30 | Stevia (Natura Greece) @ 90 g/L |
| 30/60 | Stevia (Natura Greece) @ 30 g/L |
| 30/30 | Stevia (Natura Greece) @ 30 g/L |
| 30 1/3 | Stevia (Natura Greece) @ 30 g/L |
| 30 1/2 | Stevia (Natura Greece) @ 30 g/L |
| 90 1/2 | Stevia (Natura Greece) @ 90 g/L |

| Infusion: | | |
|---|---|---|
| Hot water | variable | mL |
| Time (tea steep) | variable | min |
| Temp (start) | 60 | ° C. |
| Temp (end) | / | ° C. |

Stevia infusion preparation:

Seven different stevia infusions were prepared (but were not fermented in this trial); 1.5 L of total infusion in each jar:

90/60-90 g/L, infused at 60° C., using all the water topping up only the amount use by the leaves and kept for 60 min. 90/30-90 g/L, infused at 60° C., using all the water topping up only the amount use by the leaves and kept for 30 min. 30/60-30 g/L, infused at 60° C., using all the water topping up only the amount use by the leaves and kept for 60 min. 30/30-30 g/L, infused at 60° C., using all the water topping up only the amount use by the leaves and kept for 30 min. 90 1/2-90 g/L, infused at 60° C., using half of the water amount and topping up the rest and kept for 60 min. 30 1/2-30 g/L, infused at 60° C., using half of the water amount and topping up the rest and kept for 60 min. 30 1/3-30 g/L, infused at 60° C., using a third of the water amount and topping up the rest and kept for 60 min.

Results:

Steviol glycosides analysis:

Full analysis is shown in Table 5.

TABLE 5
HPLC analysis of steviol glycosides in Example 5

| | | | | | | | | | | Total | | | | | |
|---|---|---|---|---|---|---|---|---|---|---|---|---|---|---|---|
| | | | | | | Dulco- | | | | Steviol | Steviol | | | | |
| | | | Stevio- | Reb | Reb | side | Rub- | Reb | Steviol- | Glyco- | Equiv- | | | Comment | |
| Jar# | Reb D | Reb A | side | F | C | A | usoside | B | bioside | sides | alents | | Bx | on taste | Smell |
| 30 1/3 | 51.7 | 986.5 | 615 | 41.8 | 219.2 | 17.4 | nd | 6.9 | 14.7 | 1953.2 | 692.4 | mg/l | / | | |
| 30 1/2 | 56.4 | 1142 | 715.6 | 49.3 | 255.5 | 22.3 | nd | 7.3 | 11 | 2259.3 | 800.4 | mg/l | 1.15 | profile similar | slightly |
| | | | | | | | | | | | | | | sweetness to | herbal but |
| | | | | | | | | | | | | | | 30/30 slightly | not so |
| | | | | | | | | | | | | | | less lingering | strong |
| | | | | | | | | | | | | | | than 30/30 | |
| | | | | | | | | | | | | | | slightly | |
| | | | | | | | | | | | | | | less intensity | |
| | | | | | | | | | | | | | | than 90/30 | |
| 90 1/2 | 128.8 | 1813.6 | 1129.8 | 82.4 | 413 | 39 | 10.1 | 14.2 | 22.1 | 3653 | 1293.6 | mg/l | 1.81 | watery not | no smell |
| | | | | | | | | | | | | | | enough | |
| | | | | | | | | | | | | | | sweet even if | |
| | | | | | | | | | | | | | | less lingering | |
| 30/30 | 65.4 | 1315.2 | 835.8 | 57 | 293.3 | 27.7 | nd | 8.4 | 12.7 | 2615.6 | 927.2 | mg/l | 1.31 | same profile | herbal but |
| 1L | | | | | | | | | | | | | | of the 90/30 | the usual |
| | | | | | | | | | | | | | | but slightly | smell of |
| | | | | | | | | | | | | | | less intense | stevia |
| | | | | | | | | | | | | | | | infusion |
| 30/60 | 63.2 | 1294.3 | 820.7 | 54.9 | 288.1 | 25.5 | nd | 7.5 | 15.2 | 2569.4 | 911.1 | mg/l | 1.32 | cooked, lot | cooked |
| 1L | | | | | | | | | | | | | | of aftertaste | smell, not |
| | | | | | | | | | | | | | | but same | much |
| | | | | | | | | | | | | | | sweetness | herbal |
| | | | | | | | | | | | | | | of 90/30 | |
| 90/30 | 251.1 | 3125.3 | 1982.2 | 134.9 | 725.2 | 61.1 | 12.8 | 24.6 | 40.6 | 6357.8 | 2250.4 | mg/l | 3.32 | sweeter and | strong |
| 1L | | | | | | | | | | | | | | herbal | herbal |
| | | | | | | | | | | | | | | aftertaste | |
| 90/60 | 236.5 | 2995.5 | 1893.9 | 131.3 | 697.2 | 64.1 | 16.8 | 29.2 | 48.2 | 6112.7 | 2166.2 | mg/l | 3.19 | less aftertaste | herbal but |
| 1L | | | | | | | | | | | | | | but quite less | in the |
| | | | | | | | | | | | | | | sweetness | norm |

### Example 6

### Fermentation of Stevia Infusions with S. Cerevisiae Yeast

Experimental parameters:

| Jar # | Raw material, supplier and dose g/L |
|---|---|
| SNSA30R | Stevia Natura SAS @ 30 g/L |
| P30 | Stevia Paraguay @ 30 g/L |
| P60 | Stevia Paraguay @ 60 g/L |

| Infusion (step 1): | | |
|---|---|---|
| Hot water | 2500 | mL |
| Time (tea steep) | 30 | min |
| Temp (start) | 60 | ° C. |
| Temp (end) | / | ° C. |
| Fermentation (step 2): | | |
| Sugar (3Bx) | 30 | g/L |
| Yeast | 1.2 | g |
| Infusion (from step 1) | 2500 | mL |
| Cold Water | 0 | mL |
| Water for Yeast | 500 | mL |

The stevia infusion was prepared and fermented (using a S. cerevisiae strain) using the method as described for Example 2.

Results:

Monitoring of fermentation reaction:

| | Jar # | Start (0 h) | Day 1 (18 h) |
|---|---|---|---|
| Brix | SNSA30R | 4.09 | 2.77 |
| | P30 | 3.85 | 2.5 |
| | P60 | 3.01 | 1.71 |
| PH | SNSA30R | 5.67 | 4.4 |
| | P30 | 5.64 | 4.48 |
| | P60 | 5.69 | 4.89 |
| Density (SG) | SNSA30R | 1.01426 | 1.00792 |
| | P30 | 1.01488 | 1.00749 |
| | P60 | 1.00851 | 1.00427 |

Steviol glycosides analysis:

| | Reb D | Reb A | Stevioside | Reb F | RebC | Dulcoside A | Rubusoside | Reb B | Steviolbioside | Total |
|---|---|---|---|---|---|---|---|---|---|---|
| SNSA | 107.286 | 1576.87 | 596.048 | 36.272 | 184.126 | 0.032 | | 19.83 | 25.767 | 2546.233 |
| 30 R | | | | | | | | | | |
| P30 | 47.242 | 990.778 | 740.306 | 24.109 | 130.667 | 0.219 | | 17.454 | 20.916 | 1971.691 |
| P60 | 57.925 | 1706.62 | 1301.776 | 48.361 | 243.38 | 8.465 | 3.283 | 45.406 | 48.24 | 3463.452 |
| SNSA | 87.991 | 1580.95 | 603.807 | 38.521 | 189.161 | 1.43 | | 20.571 | 28.159 | 2550.587 |
| 30 R | | | | | | | | | | |
| 18H* | | | | | | | | | | |
| P30 | 26.299 | 989.337 | 741.89 | 25.601 | 133.538 | 1.148 | 3.029 | 19.234 | 24.644 | 1964.72 |
| 18H* | | | | | | | | | | |
| P60 | 64.298 | 1723.04 | 1318.447 | 49.597 | 246.173 | 5.88 | 11.606 | 42.213 | 46.62 | 3507.874 |
| 18H* | | | | | | | | | | |

*after 18H fermentation

### Example 7

### Screening of Fermentation Microorganisms

Small samples (15 ml) of a 30 g/L stevia infusion, prepared as described herein, were supplemented with 30-50 g/L glucose and then inoculated with various fermentation microorganisms (see Table 7-1 below) and incubated in 100 ml shake flasks at 28° C. with shaking. Microorganism biomass was freshly generated on a small scale to serve as inoculum in the infusion. Duration of fermentation was 48 hours, with the aim of completely consuming the sugar. Harvested samples were centrifuged in 15 ml or 50 ml PP tubes (e.g. Falcon, Corning) at room temperature for 10 minutes (e.g. at 4500 rpm in a Thermo Scientific Multifuge X3R). The supernatant was transferred to fresh PP tubes and pasteurized in a 75° C. waterbath (e.g. SW22, Julabo) for 20 min. The experiments were repeated twice.

Fermented samples were diluted 1:10 with mineral water. A panel of tasters ranked them on a liking scale. The most successful fermentations (i.e. those providing the most liked taste results) were repeated on a larger scale (500mL) to validate the results and provide samples for HPLC analysis (Table 7-2).

HPLC method I: HPLC analysis was performed using a Phenomenex Synergi column: 2.5 μm Hydro-RP 100 A, 100*2; Solvent A: 0.04% acetic acid; Solvent B: methanol +0.04% acetic acid; Flow: isocratic 50%B with 0.25 ml/min. Total runtime was 30 min. MS detection in negative mode 500-1200 m/z; MS fragmentation in negative mode Bruker AmazonSL lonTrap (auto or manual); Samples are diluted 1:10 in mobile phase and filtrated (hPTFE 0,22 μm) prior to injection; Injection volume: 10 μl.

Spectra and fragmentation patterns were compared against standards for Reb A, Reb D and Reb I. Retention times for these reference compounds were 21 min (m/z 965.47), 8.4 min (m/z 1127.71) and 20 min (m/z 1127.69), respectively.

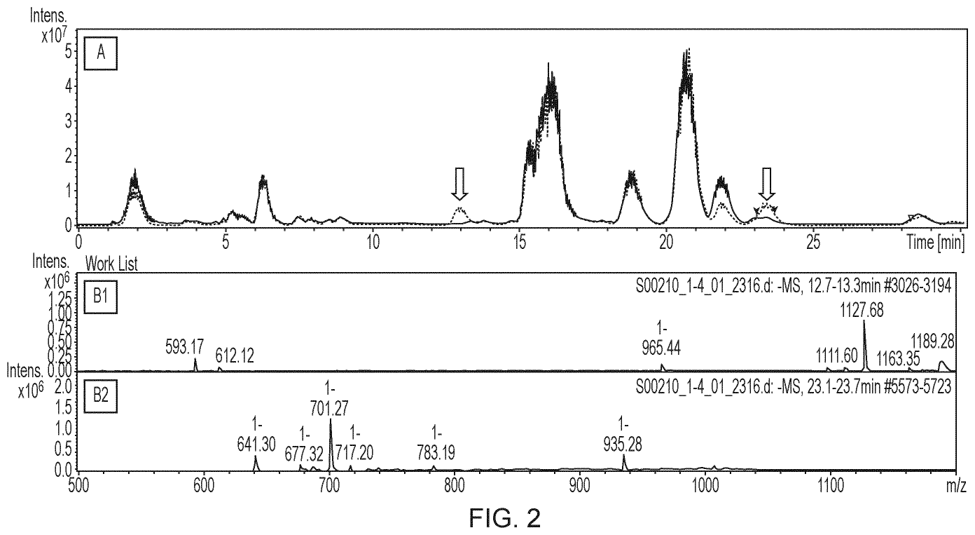

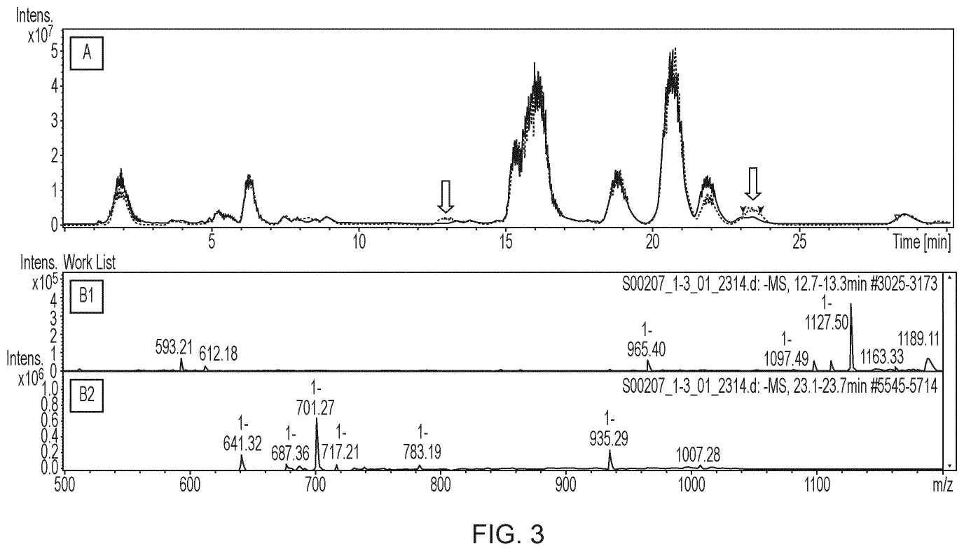

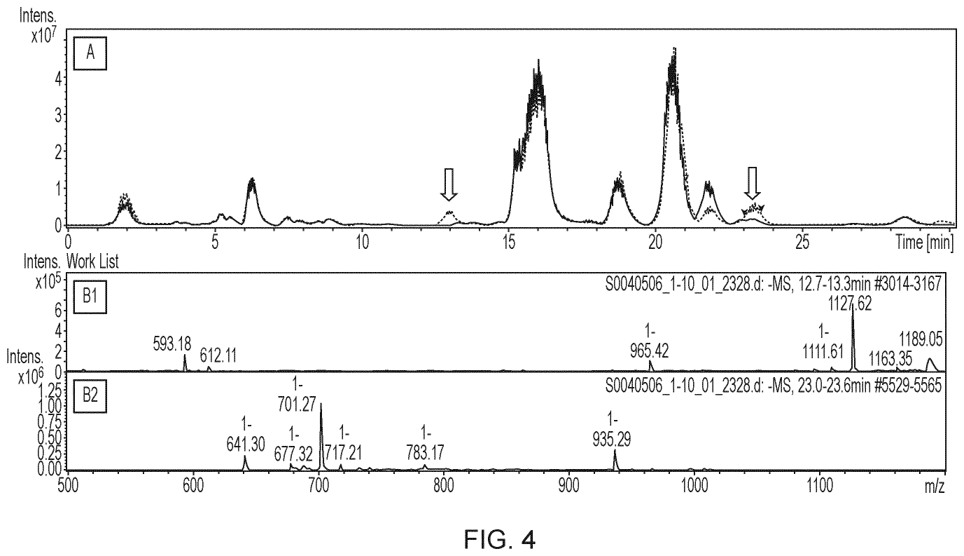

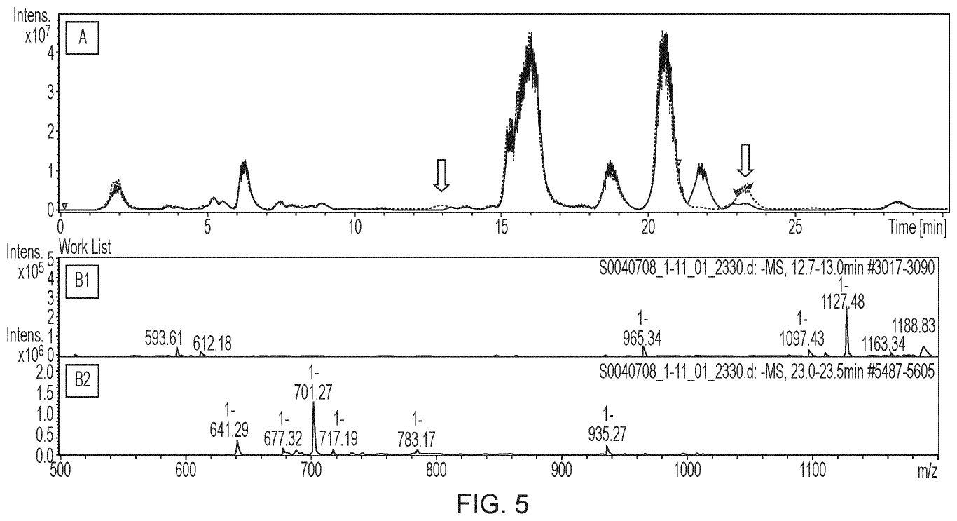

Exemplary HPLC spectra for fermented samples according to the invention are shown in FIGS. 2 to 6.

Sensory results (trained panel):

Sensory profiles for exemplary fermented samples are shown in FIG. 7.

Reference sample (non fermented): green appearance (the darkest), ash/woody notes (aroma and flavour), no fermented notes (aroma and flavour).

Sample 10 (Zygosaccharomyces rouxii): very light in colour, mild ash/woody notes (aroma), fermented notes (aroma). The flavour is not very woody and has mild fermented notes, astringent mouthfeel.

Sample 19 (Meyerozyma guilliermondii): light colour, ash/woody and fermented aroma notes, fermented (the most) and woody flavour, astringent mouthfeel.

TABLE 7-1
screening

| | Residual | | | | |
|---|---|---|---|---|---|
| Sample | glucose g/L | | Type of | | Strain |
| # | Run 1 | Run 2 | sample | Microorganism | # |
| REF | 50 | 50 | control-no | — | — |
| | | | microbial | | |
| | | | inoculum | | |
| 01 | 10 | 7.75 | fermented | Kluyveromyces lactis | A |
| 02 | 2 | 0.50 | fermented | Kluyveromyces lactis | B |
| 03 | 0 | 0 | fermented | Kluyveromyces marxianus | C |
| 04 | 2 | 6.70 | fermented | Kluyveromyces marxianus | D |
| 05 | 0 | 0 | fermented | Kluyveromyces marxianus | E |
| 06 | 0 | 30.00 | fermented | Zygosaccharomaces rouxii | F |
| 07 | 12 | 11.50 | fermented | Pichia membranifaciens | G |
| 08 | 0 | 0 | fermented | Kluyveromyces marxianus | H |
| 09 | 0 | 0 | fermented | Kluyveromyces marxianus | J |
| 10 | 0 | 3.00 | fermented | Zygosaccharomyces rouxii | K |
| 11 | 0 | 0 | fermented | Kluyveromyces marxianus | L |
| 12 | 0 | 25.00 | fermented | Kluyveromyces marxianus | M |
| 13 | 0 | 0 | fermented | Kluyveromyces marxianus | N |
| 14 | 12 | 13.50 | fermented | Kluyveromyces marxianus | P |
| 15 | 0 | 0 | fermented | Cyberlindnera jadinii | Q |
| 16 | 0 | 8.50 | fermented | Meyerozyma guilliermondii | R |
| 17 | 6 | 0 | fermented | Meyerozyma guilliermondii | S |
| 18 | 0 | 0 | fermented | Meyerozyma guilliermondii | T |
| 19 | 3 | 0 | fermented | Meyerozyma guilliermondii | U |

TABLE 7-2
scale-up

| | Residual | | | |
|---|---|---|---|---|
| | glucose | Type | | Strain |
| Sample # | g/L | of sample | Microorganism | # |
| REF | 50 | control-no | — | — |
| | | microbial | | |
| | | inoculum | | |
| 01_02 | 30.00 | fermented | Zygosaccharomaces rouxii | F |
| 03_04 | 7.7 | fermented | Pichia membranifaciens | G |
| 05_06 | 0.44 | fermented | Zygosaccharomyces rouxii | K |
| 07_08 | 0.00 | fermented | Cyberlindnera jadinii | Q |
| 09_10 | 5.00 | fermented | Meyerozyma guilliermondii | U |

### Example 7B

### Quantification of Steviol Glycosides of an Exemplary Sample

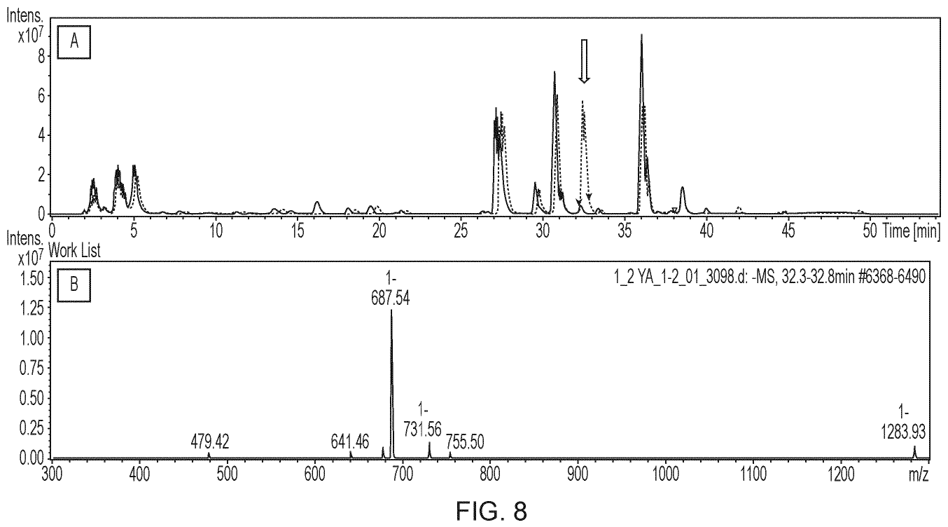

A stevia infusion (30 g/L) volume of 15m1, supplemented with 30 g/L glucose, was inoculated with strain # K (freshly generated biomass) and incubated in 100mIshake flasks at 28° C. with shaking (sample # S015B). Duration of fermentation was 48 hours, with the aim of completely consuming the sugar. Harvesting was done as in example 7A. Sample and control were analysed targeting a larger set of standards (qualitative analysis, FIG. 8) and quantitative assessment was performed (Table 7-3).

HPLC method II: HPLC analysis was performed using a Kinetex C18 2.6 μm 150*2,1 mm column; Solvent A: 0.1% formic acid Solvent B: AcN +0.1% formic acid; Flow: binary gradient 0.2 ml/min starting with 20%B.

Total runtime was 54 min. MS detection in negative mode 300-1300 m/z. Samples are diluted 1:10 in mobile phase (80%A/20%B) and filtrated (hPTFE 0,22 μm) prior to injection; Injection volume: 10p1

Spectra and fragmentation patterns were compared against standards for Reb E, Reb D, Reb M, Reb I, Reb A, Reb F, Reb C, Reb B as well as Stevioside, Dulcoside A, Rubusoside, Steviobioside.

HPLC spectrum for fermented sample # S015B is shown in FIG. 8.

Analytical results:

TABLE 7-3
Exemplary Steviol glycosides [ppm] analysis sample #S015B*

| | | | | | Stevio- | | | | Dulco- | | | Rubu- | Stevio- | |
|---|---|---|---|---|---|---|---|---|---|---|---|---|---|---|
| | RebA | RebB | RebC | RebD | side | RebF | RebM | RebN | sideA | RebI | RebG | sosice | bioside | RebE |
| fermented | 2434.7 | 238.3 | 236.6 | 3.0 | 498.1 | 53.9 | 32.7 | 32.2 | 11.2 | 1.7 | 0.0 | 271.9 | 33.1 | 0.2 |
| (sample | | | | | | | | | | | | | | |
| #S015B) | | | | | | | | | | | | | | |
| unfermented | 3066.4 | 356.5 | 312.6 | 107.7 | 1156.8 | 72.0 | 40.4 | 40.3 | 14.3 | 12.4 | 76.4 | 13.1 | 105.2 | 19.2 |

*Methodology: Food and Chemical Toxicology 41 (2003) 359-374

### Example 8

### Screening of Fermentation Microorganisms

Small samples (15 ml) of a 60 g/L stevia infusion, prepared as described herein, were supplemented with 30 g/L glucose and then inoculated with various fermentation microorganisms and incubated in 100 ml shake flasks. Microbial strains were cultivated on a small scale in shake flasks, harvested after 2 days, suspended in spent medium and subsequently served as inoculum in the infusion. Duration of fermentation step was 48 hours. Harvesting was done as in example 7A.

Experimental parameters:

| | | | Medium | | | | | | | | | |
|---|---|---|---|---|---|---|---|---|---|---|---|---|
| Strain | | | 30 ml Medium, | | | resuspend in | | | | | | |
| Culture | Species | Strain | 300 ml shakeflask | Sugar g/L | | spent medium | Temp | rpm | Gas | | | |
| MA.A1 | Pichia | S-I | YPD | Glucose | 15 | 2 | 28° C. | 180 | aerobic | | | |
| | membranifaciens | | | | | | | | | | | |
| MA.A2 | Cyberlindnera | S-II | YPD | Glucose | 15 | 2 | 28° C. | 180 | aerobic | | | |
| | jadinii | | | | | | | | | | | |
| MA.A3 | Meyerozyma | S-III | YPD | Glucose | 15 | 2 | 28° C. | 180 | aerobic | | | |
| | guilliermondii | | | | | | | | | | | |
| MA.B1 | Lactobacillus | S-IV | MRS | Glucose | 20 | 2 | 37° C. | 0 | anaerobic | | | |
| | acidophilus | | | | | | | | | | | |
| MA.B2 | Lactobacillus | S-IV | MRS | Glucose | 20 | 2 | 37° C. | 0 | anaerobic | | | |
| | acidophilus | | | | | | | | | | | |
| MA.B3 | Lactobacillus | S-IV | MRS | Glucose | 20 | 2 | 37° C. | 0 | anaerobic | | | |
| | acidophilus | | | | | | | | | | | |
| MA.C1 | Lactobacillus | S-V | MRS | Glucose | 20 | 2 | 37° C. | 0 | anaerobic | | | |
| | delbrueckil | | | | | | | | | | | |
| MA.C2 | Lactobacillus | S-V | MRS | Glucose | 20 | 2 | 37° C. | 0 | anaerobic | | | |
| | delbrueckil | | | | | | | | | | | |
| MA.C3 | Lactobacillus | S-V | MRS | Glucose | 20 | 2 | 37° C. | 0 | anaerobic | | | |
| | delbrueckil | | | | | | | | | | | |
| MA.D1 | Lactobacillus | S-VI | MRS | Glucose | 20 | 2 | 28° C. | 180 | aerobic | | | |
| | fructivorans | | | | | | | | | | | |
| MA.D2 | Lactobacillus | S-VI | MRS | Glucose | 20 | 2 | 28° C. | 180 | aerobic | | | |
| | fructivorans | | | | | | | | | | | |
| MA.D3 | Lactobacillus | S-VI | MRS | Glucose | 20 | 2 | 28° C. | 180 | aerobic | | | |
| | fructivorans | | | | | | | | | | | |
| MA.E1 | Lactobacillus | S-VII | MRS | Glucose | 20 | 2 | 28° C. | 180 | aerobic | | | |
| | sakei | | | | | | | | | | | |
| MA.E2 | Lactobacillus | S-VII | MRS | Glucose | 20 | 2 | 28° C. | 180 | aerobic | | | |
| | sakei | | | | | | | | | | | |
| MA.F1 | Lactococcus | S-VIII | MRS | Glucose | 20 | 2 | 28° C. | 180 | aerobic | | | |
| | raffinolactis | | | | | | | | | | | |
| MA.F2 | Lactococcus | S-VIII | MRS | Glucose | 20 | 2 | 28° C. | 180 | aerobic | | | |
| | raffinolactis | | | | | | | | | | | |
| | | | | | Infusion | | | Biomass | | | | |
| | Bacterium | | Yeast | | Stevia | | | Suspension (μl) | | Temp | | |
| Sample | Name | Strain | Name | Strain | (g/L) | sugar [g/L] | Bacterium | Yeast | | [° C.] | rpm | Gas |
| MA1 | Lactobacillus | S-IV | — | — | 60 | Lactose | 30 | 2000 | 0 | 37 | 40 | anaerobic |
| | acidophilus | | | | | | | | | | | |
| MA2 | Lactobacillus | S-V | — | — | 60 | Lactose | 30 | 2000 | 0 | 37 | 40 | anaerobic |
| | delbrueckii | | | | | | | | | | | |
| MA3 | Lactobacillus | S-VI | Pichia | S-I | 60 | Glucose | 30 | 1000 | 200 | 28 | 180 | aerobic |
| | fructivorars | | membranifaciens | | | | | | | | | |
| MA4 | Lactobacillus | S-VI | Pichia | S-I | 60 | Glucose | 30 | 1000 | 50 | 28 | 180 | aerobic |
| | fructivorars | | membranifaciens | | | | | | | | | |
| MA5 | Lactobacillus | S-IV | Cyberlindnera | S-II | 60 | Glucose | 30 | 1000 | 200 | 37 | 40 | anaerobic |
| | acidophilus | | jadinii | | | | | | | | | |
| MA6 | Lactobacillus | S-V | Cyberlindnera | S-II | 60 | Glucose | 30 | 1000 | 200 | 37 | 40 | anaerobic |
| | delbrueckii | | jadinii | | | | | | | | | |
| MA7 | Lactobacillus | S-IV | Meyerozyma | S-III | 60 | Glucose | 30 | 1000 | 200 | 37 | 40 | anaerobic |
| | acidophilus | | guilliermondii | | | | | | | | | |
| MA8 | Lactobacillus | S-V | Meyerozyma | S-III | 60 | Glucose | 30 | 1000 | 200 | 37 | 40 | anaerobic |
| | delbrueckii | | guilliermondii | | | | | | | | | |
| MA9 | Lactobacillus | S-VI | Meyerozyma | S-III | 60 | Glucose | 30 | 1000 | 200 | 28 | 180 | aerobic |
| | fructivorars | | guilliermondii | | | | | | | | | |
| MA10 | Lactobacillus | S-VII | Meyerozyma | S-III | 60 | Glucose | 30 | 1000 | 200 | 28 | 180 | aerobic |
| | sakei | | guilliermondii | | | | | | | | | |
| MA11 | Lactococcus | S-VIII | Meyerozyma | S-III | 60 | Glucose | 30 | 1000 | 200 | 28 | 180 | aerobic |
| | raffinolactis | | guilliermondii | | | | | | | | | |

Analytical results:

HPLC method I: Sugar, acid analytics
Column: Rezex™ ROA-Organic Acid H+(8%), 300×4.6 mm
Solvent A: 0.1% (v/v) trifluoroacetic acid (TFA)
Flow: isocratic
Total runtime: 30 min
Detection by DAD (210 nm) and RID
Samples are diluted 2- or 5-fold (depending on expected metabolite concentrations) with appropriate volumes of 2% (v/v) and water to a final concentration of 1% (v/v) TFA and filtered (hPTFE 0.22 μm) prior to injection
Injection volume: 10 μl
Target analytes: acetic acid, formic acid, fructose, glucose, glycerol, lactic acid and succinic acid

| | Glucose | Lactose | Glycerol | Lactate | |
|---|---|---|---|---|---|
| Sample # | [g/L] | [g/L] | [g/L] | [g/L] | PH |
| MA1 | 0.00 | 11.02 | 3.84 | 6.22 | 3.55 |
| MA2 | 0.00 | 12.29 | 4.47 | 7.20 | 3.43 |
| MA3 | 0.01 | 0.00 | 2.90 | 2.59 | 3.79 |
| MA4 | 1.14 | 0.00 | 2.67 | 2.33 | 3.71 |
| MA5 | 0.15 | 0.00 | 3.76 | 5.41 | 3.52 |
| MA6 | 0.00 | 0.00 | 3.44 | 5.22 | 3.55 |
| MA7 | 12.18 | 0.00 | 4.08 | 6.76 | 3.43 |
| MA8 | 12.27 | 0.00 | 4.06 | 6.67 | 3.44 |
| MA9 | 0.00 | 0.00 | 2.16 | 3.65 | 3.75 |
| MA10 | 0.00 | 0.00 | 2.72 | 3.95 | 3.7 |
| MA11 | 0.00 | 0.00 | 0.00 | 0.00 | 6.23 |
| MA12 | 15.35 | 0.00 | 2.10 | 2.39 | 4.08 |

HPLC method II: Qualitative Steviolglycoside analytics
Column: Kinetex C18 2.6 μm 150*2.1 mm
Solvent A: 0.1% formic acid
Solvent B: AcN+0,1% formic acid
Flow: binary gradient 0.2 ml/min starting with 20%B
Total runtime: 54 min
MS detection in negative mode 300-1300 m/z
Samples are diluted 1:10 in mobile phase (80%A/20%B) and filtrated (hPTFE 0,22 μm) prior to injection
Injection volume: 10 μl
Analyte target: Reb E, D, M, I, A, F, C, B as well as Stevioside, Dulcoside A, Rubusoside, Steviobioside

| | | | | MA3 | | | MA4 | | |
|---|---|---|---|---|---|---|---|---|---|
| | RT | CTLMA Glucose | | | | delta | | | delta |
| Analyte | [min] | Chromatogram | Area | Area | delta Area | Area % | Area | delta Area | Area % |
| RebE | 14.3 | EIC 965.77 -All MS | 118840896 | 173911168 | 55070272 | 46.34 | 182571200 | 63730304 | 53.63 |
| RebD | 15.9 | EIC 1127.91 -All MS | 216863408 | 291269824 | 74406416 | 34.31 | 323678240 | 106814832 | 49.25 |
| RebM | 17.8 | EIC 1290.01 -All MS | 25496538 | 37165300 | 11668762 | 45.77 | 42052952 | 16556414 | 64.94 |
| RebI | 26.2 | EIC 1127.91 -All MS | 5055771 | 3530707 | −1525064 | −30.16 | 6747964 | 1692193 | 33.47 |
| RebA | 26.9 | EIC 965.77 -All MS | 2403748096 | 2433838592 | 30090496 | 1.25 | 2511722496 | 107974400 | 4.49 |
| Stevioside | 27.3 | EIC 849.63 -All MS | 3471461120 | 3385091584 | −86369536 | −2.49 | 3575515136 | 104054016 | 3.00 |
| RebF | 29.4 | EIC 935.72 -All MS | 705598592 | 881053760 | 175455168 | 24.87 | 943990144 | 238391552 | 33.79 |
| RebC | 30.5 | EIC 949.75 -All MS | 2788458496 | 3132422912 | 343964416 | 12.34 | 3232423168 | 443964672 | 15.92 |
| Dulcoside A | 31 | EIC 833.62 -All MS | 165847904 | 218647424 | 52799520 | 31.84 | 243414560 | 77566656 | 46.77 |
| Rubusoside | 32.1 | EIC 687.51 -All MS | 305767872 | 283074944 | −22692928 | −7.42 | 290426112 | −15341760 | −5.02 |
| RebB | 35.9 | EIC 803.65 -All MS | 458243680 | 516187168 | 57943488 | 12.64 | 585368320 | 127124640 | 27.74 |
| Steviolbioside | 36.2 | EIC 641.49 -All MS | 338164416 | 371549696 | 33385280 | 9.87 | 395571360 | 57406944 | 16.98 |
| | | | | MA5 | | | MA6 | | |
| | RT | CTLMA Glucose | | | | delta | | | delta |
| Analyte | [min] | Chromatogram | Area | Area | delta Area | Area % | Area | delta Area | Area % |
| RebE | 14.3 | EIC 965.77 -All MS | 118840896 | 130134032 | 11293136 | 9.50 | 137113936 | 18273040 | 15.38 |
| RebD | 15.9 | EIC 1127.91 -All MS | 216863408 | 254554672 | 37691264 | 17.38 | 234558512 | 17695104 | 8.16 |
| RebM | 17.8 | EIC 1290.01 -All MS | 25496538 | 7768806 | −17727732 | −69.53 | 24318018 | −1178520 | −4.62 |
| RebI | 26.2 | EIC 1127.91 -All MS | 5055771 | 13411601 | 8355830 | 165.27 | 17822552 | 12766781 | 252.52 |
| RebA | 26.9 | EIC 965.77 -All MS | 2403748096 | 2429087488 | 25339392 | 1.05 | 2357415680 | −46332416 | −1.93 |
| Stevioside | 27.3 | EIC 849.63 -All MS | 3471461120 | 3350584064 | −120877056 | −3.48 | 3263184384 | −208276736 | −6.00 |
| RebF | 29.4 | EIC 935.72 -All MS | 705598592 | 757235776 | 51637184 | 7.32 | 697186496 | −8412096 | −1.19 |
| RebC | 30.5 | EIC 949.75 -All MS | 2788458496 | 2968667136 | 180208640 | 6.46 | 2819449344 | 30990848 | 1.11 |
| Dulcoside A | 31 | EIC 833.62 -All MS | 165847904 | 188527200 | 22679296 | 13.67 | 189754176 | 23906272 | 14.41 |
| Rubusoside | 32.1 | EIC 687.51 -All MS | 305767872 | 344994528 | 39226656 | 12.83 | 322388512 | 16620640 | 5.44 |
| RebB | 35.9 | EIC 803.65 -All MS | 458243680 | 499351200 | 41107520 | 8.97 | 450058240 | −8185440 | −1.79 |
| Steviolbioside | 36.2 | EIC 641.49 -All MS | 338164416 | 341210784 | 3046368 | 0.90 | 333605696 | −4558720 | −1.35 |
| | | | | MA7 | | | MA8 | | |
| | RT | CTLMA Glucose | | | | delta | | | delta |
| Analyte | [min] | Chromatogram | Area | Area | delta Area | Area % | Area | delta Area | Area % |
| RebE | 14.3 | EIC 965.77 -All MS | 118840896 | 105447792 | −13393104 | −11.27 | 111568408 | −7272488 | −6.12 |
| RebD | 15.9 | EIC 1127.91 -All MS | 216863408 | 187994496 | −28868912 | −13.31 | 201641680 | −15221728 | −7.02 |
| RebM | 17.8 | EIC 1290.01 -All MS | 25496538 | 29389772 | 3893234 | 15.27 | 32791622 | 7295084 | 28.61 |
| RebI | 26.2 | EIC 1127.91 -All MS | 5055771 | 8223428 | 3167657 | 62.65 | 2684789 | −2370982 | −46.90 |
| RebA | 26.9 | EIC 965.77 -All MS | 2403748096 | 2338661632 | −65086464 | −2.71 | 2344601600 | −59146496 | −2.46 |
| Stevioside | 27.3 | EIC 849.63 -All MS | 3471461120 | 3098226688 | −373234432 | −10.75 | 3175481600 | −295979520 | −8.53 |
| RebF | 29.4 | EIC 935.72 -All MS | 705598592 | 634420544 | −71178048 | −10.09 | 652564928 | −53033664 | −7.52 |
| RebC | 30.5 | EIC 949.75 -All MS | 2788458496 | 2672513280 | −115945216 | −4.16 | 2773452800 | −15005696 | −0.54 |
| Dulcoside A | 31 | EIC 833.62 -All MS | 165847904 | 157357360 | −8490544 | −5.12 | 191119392 | 25271488 | 15.24 |
| Rubusoside | 32.1 | EIC 687.51 -All MS | 305767872 | 395492736 | 89724864 | 29.34 | 445701824 | 139933952 | 45.76 |
| RebB | 35.9 | EIC 803.65 -All MS | 458243680 | 433592320 | −24651360 | −5.38 | 438780480 | −19463200 | −4.25 |
| Steviolbioside | 36.2 | EIC 641.49 -All MS | 338164416 | 280241536 | −57922880 | −17.13 | 313730464 | −24433952 | −7.23 |
| | | | | MA9 | | | MA10 | | |
| | RT | CTLMA Glucose | | | | delta | | | delta |
| Analyte | [min] | Chromatogram | Area | Area | delta Area | Area % | Area | delta Area | Area % |
| RebE | 14.3 | EIC 965.77 -All MS | 118840896 | 152601872 | 33760976 | 28.41 | 114700752 | −4140144 | −3.48 |
| RebD | 15.9 | EIC 1127.91 -All MS | 216863408 | 293296480 | 76433072 | 35.24 | 210775696 | −6087712 | −2.81 |
| RebM | 17.8 | EIC 1290.01 -All MS | 25496538 | 37296912 | 11800374 | 46.28 | 20888580 | −4607958 | −18.07 |
| RebI | 26.2 | EIC 1127.91 -All MS | 5055771 | 15741744 | 10685973 | 211.36 | 3196195 | −1859576 | −36.78 |
| RebA | 26.9 | EIC 965.77 -All MS | 2403748096 | 2582359808 | 178611712 | 7.43 | 2333189632 | −70558464 | −2.94 |
| Stevioside | 27.3 | EIC 849.63 -All MS | 3471461120 | 3521027072 | 49565952 | 1.43 | 3285468160 | −185992960 | −5.36 |
| RebF | 29.4 | EIC 935.72 -All MS | 705598592 | 903390464 | 197791872 | 28.03 | 686232064 | −19366528 | −2.74 |
| RebC | 30.5 | EIC 949.75 -All MS | 2788458496 | 3215042048 | 426583552 | 15.30 | 2708335360 | −80123136 | −2.87 |
| Dulcoside A | 31 | EIC 833.62 -All MS | 165847904 | 226486512 | 60638608 | 36.56 | 171326832 | 5478928 | 3.30 |
| Rubusoside | 32.1 | EIC 687.51 -All MS | 305767872 | 493957696 | 188189824 | 61.55 | 373185984 | 67418112 | 22.05 |
| RebB | 35.9 | EIC 803.65 -All MS | 458243680 | 561528000 | 103284320 | 22.54 | 433017664 | −25226016 | −5.50 |
| Steviolbioside | 36.2 | EIC 641.49 -All MS | 338164416 | 390759616 | 52595200 | 15.55 | 279482176 | −58682240 | −17.35 |
| | | | | MA11 | | | MA1 | | |
| | RT | CTLMA Glucose | | | | delta | | | delta |
| Analyte | [min] | Chromatogram | Area | Area | delta Area | Area % | Area | delta Area | Area % |
| RebE | 14.3 | EIC 965.77 -All MS | 118840896 | 138725744 | 19884848 | 16.73 | 180992656 | −4367616 | −2.36 |
| RebD | 15.9 | EIC 1127.91 -All MS | 216863408 | 244705056 | 27841648 | 12.84 | 309578080 | 4667808 | 1.53 |
| RebM | 17.8 | EIC 1290.01 -All MS | 25496538 | 40670944 | 15174406 | 59.52 | 39330088 | −8121192 | −17.11 |
| RebI | 26.2 | EIC 1127.91 -All MS | 5055771 | 4050426 | −1005345 | −19.89 | 13217289 | 8366603 | 172.48 |
| RebA | 26.9 | EIC 965.77 -All MS | 2403748096 | 2408734976 | 4986880 | 0.21 | 2476339200 | 45306368 | 1.86 |
| Stevioside | 27.3 | EIC 849.63 -All MS | 3471461120 | 3532585216 | 61124096 | 1.76 | 3459366656 | −173717760 | −4.78 |
| RebF | 29.4 | EIC 935.72 -All MS | 705598592 | 758464576 | 52865984 | 7.49 | 874148480 | −2448256 | −0.28 |
| RebC | 30.5 | EIC 949.75 -All MS | 2788458496 | 2929282560 | 140824064 | 5.05 | 3161312256 | −261122048 | −7.63 |
| Dulcoside A | 31 | EIC 833.62 -All MS | 165847904 | 233350144 | 67502240 | 40.70 | 206232352 | −27650224 | −11.82 |
| Rubusoside | 32.1 | EIC 687.51 -All MS | 305767872 | 593589312 | 287821440 | 94.13 | 366702304 | 3952288 | 1.09 |
| RebB | 35.9 | EIC 803.65 -All MS | 458243680 | 660848384 | 202604704 | 44.21 | 635858432 | 42884160 | 7.23 |
| Steviolbioside | 36.2 | EIC 641.49 -All MS | 338164416 | 529575456 | 191411040 | 56.60 | 426144640 | 52184800 | 13.95 |
| | | | | MA2 | | | | | |
| | RT | CTLMA Lactose | | | | delta | | | |
| Analyte | [min] | Chromatogram | Area | Area | delta Area | Area % | | | |
| RebE | 14.3 | EIC 965.77 -All MS | 185360272 | 175762640 | −9597632 | −5.18 | | | |
| RebD | 15.9 | EIC 1127.91 -All MS | 304910272 | 296507200 | −8403072 | −2.76 | | | |
| RebM | 17.8 | EIC 1290.01 -All MS | 47451280 | 49408896 | 1957616 | 4.13 | | | |
| RebI | 26.2 | EIC 1127.91 -All MS | 4850686 | 8732304 | 3881618 | 80.02 | | | |
| RebA | 26.9 | EIC 965.77 -All MS | 2431032832 | 2474366976 | 43334144 | 1.78 | | | |
| Stevioside | 27.3 | EIC 849.63 -All MS | 3633084416 | 3388836096 | −244248320 | −6.72 | | | |
| RebF | 29.4 | EIC 935.72 -All MS | 876596736 | 850696576 | −25900160 | −2.95 | | | |
| RebC | 30.5 | EIC 949.75 -All MS | 3422434304 | 3162701312 | −259732992 | −7.59 | | | |
| Dulcoside A | 31 | EIC 833.62 -All MS | 233882576 | 193511808 | −40370768 | −17.26 | | | |
| Rubusoside | 32.1 | EIC 687.51 -All MS | 362750016 | 294320576 | −68429440 | −18.86 | | | |
| RebB | 35.9 | EIC 803.65 -All MS | 592974272 | 571269632 | −21704640 | −3.66 | | | |
| Steviolbioside | 36.2 | EIC 641.49 -All MS | 373959840 | 365399744 | −8560096 | −2.29 | | | |

Sensory results:

Fermented samples were applied in a beverage composition as follows:
Prepare a concentrated base of the drink that will be diluted 1:4 (1 part of base for 4 parts of water).
Add the ingredient at 10 g/L
Top up with the remaining part of water.
Blackcurrant drink:
BASE INGREDIENTS:
Water, Blackcurrant Juice from Concentrate (6%), Sugar, Thickener (Polydextrose), Acidity Regulator (Sodium Gluconate), Extracts of Carrot and Hibiscus, Vitamin C, Natural Blackcurrant Flavourings, Acid (Citric Acid).

Fermented samples were tasted by 4 trained tasters rating each descriptor from 1 to 5. A full sugar beverage was applied as positive benchmark (FS=Full sugar) and a beverage with artificial sweeteners served as negative control (H=Base with artificial sweeteners).

| Sample Number | FS | H | 21/1 | 21/2 | 21/5 | 21/6 | 21/7 | 21/8 | 21/9 | 21/10 | 21/11 |
|---|---|---|---|---|---|---|---|---|---|---|---|
| Aroma | | | | | | | | | | | |
| Intensity | 5 | 3 | | | | | | | | | |
| Blackcurrant | 4 | 5 | 1 | 2 | 0 | 3 | 4 | 1 | 2 | 0 | 0 |
| Alcohol | 0 | 0 | 0 | 0 | 0 | 2 | 0 | 0 | 0 | 0 | 0 |
| Mouldy | 0 | 0 | 3 | 1 | 5 | 1 | 0 | 3 | 1 | 3 | 5 |
| | | | | herbal/tea | hearth/cheesy | | | dusty/musty | dusty/musty | cheesy | butyric/cheesy |
| Taste | | | | | | | | | | | |
| Sweetness | 4 | 5 | 2 | 2 | 3 | 2 | 4 | 2 | 2 | 3 | 1 |
| Sourness | 3 | 3 | 1 | 3 | 2 | 1 | 3 | 4 | 2 | 1 | 1 |
| Flavour | | | | | | | | | | | |
| Blackcurrant | 4.5 | 5 | 1 | 2 | 2 | 1 | 3.5 | 2 | 0 | 2 | |
| Fermented | 0 | 0 | 0 | 0 | 0 | 3 | 0 | 0 | 1 | 0 | |
| Mouldy/Yeast | 0 | 0 | 3 | 0 | 3 | 1 | 0 | 2 | 2 | 2 | |
| Herbal | 0 | 1 | 4 | 3 | 0 | 1 | 0.5 | 2 | 0 | 0 | |
| Yoghurt/Cheese | | | | 0 | 3 | 1 | 0 | 0 | 1 | 0 | 5 |
| Mouthfeel | 4.5 | 5 | 1 | 1 | 3 | 0 | 2 | 1 | 1 | 2 | 3 |
| Aftertaste | | | | | | | | | | | |
| Artificialness | 0 | 5 | 0 | 3 | 1 | 1 | 0 | 2 | 1 | 2 | |
| Linguerings | 0 | 5 | 1 | 0 | 2 | 0 | 1 | 3 | 2 | 3 | |
| Astringency | 1 | 2.5 | 3 | 2 | 1 | 1 | 1 | 0 | 1 | 0 | |
| | | | dusty | tea, candy | cherry | watery | flavour | | overriped fruit | off balance | |
| | | | aroma | | | | linguering | | | | |
| | | | | | | | not bad | | | | |

most balanced
too cheesy

### Example 9

### Screening of Fermentation Microorganisms

Small samples (50 ml) of a 60 g/L stevia infusion, prepared as described herein, were supplemented with 30 g/L sugar and then inoculated with various fermentation microorganisms and incubated in 300 ml shake flasks at 28° C. with shaking. Microbial strains were cultivated on a small scale in shake flasks, harvested after 2 days, suspended in spent medium and subsequently served as inoculum in the infusion. Duration of fermentation step was 3 days.

Harvesting was done as in example 7A.

Experimental parameters:

| | | | | | | resuspend in | | | | | | |
|---|---|---|---|---|---|---|---|---|---|---|---|---|
| Strain | | | Medium | | | spent medium | | | | | | |
| Culture | Species | Strain | | Sugar g/L | | [ml] | Temp | rpm | Gas | | | |
| | | | 50 ml Medium, | | | | | | | | | |
| | | | 500 ml shakeflask | | | | | | | | | |
| MB.A1 | Meyerozyma | S-III | YPD | Glucose | 15 | 3 | 28° C. | 180 | aerobic | | | |
| | guilliermondii | | | | | | | | | | | |
| MB.A2 | Meyerozyma | S-IX | YPD | Glucose | 15 | 3 | 28° C. | 180 | aerobic | | | |
| | guilliermondii | | | | | | | | | | | |
| MB.A3 | Meyerozyma | S-X | YPD | Glucose | 15 | 3 | 28° C. | 180 | aerobic | | | |
| | guilliermondii | | | | | | | | | | | |
| | | | 150 ml Medium, | | | | | | | | | |
| | | | 300 ml shakeflask | | | | | | | | | |
| MB.B | Lactobacillus | S-IV | MRS | Glucose | 20 | 5 | 37° C. | 0 | anaerobic | | | |
| | acidophilus | | | | | | | | | | | |
| MB.C | Lactobacillus | S-IV | MRS | Glucose | 20 | 5 | 37° C. | 0 | anaerobic | | | |
| | acidophilus | | | | | | | | | | | |
| MB.D | Lactobacillus | S-XI | MRS | Glucose | 20 | 5 | 37° C. | 0 | anaerobic | | | |
| | acidophilus | | | | | | | | | | | |
| MB.E | Lactobacillus | S-XI | MRS | Glucose | 20 | 5 | 37° C. | 0 | anaerobic | | | |
| | acidophilus | | | | | | | | | | | |
| MB.F | Lactobacillus | S-XII | MRS | Glucose | 20 | 5 | 37° C. | 0 | anaerobic | | | |
| | acidophilus | | | | | | | | | | | |
| MB.G | Lactobacillus | S-XII | MRS | Glucose | 20 | 5 | 37° C. | 0 | anaerobic | | | |
| | acidophilus | | | | | | | | | | | |
| MB.H | Lactobacillus | S-XIII | MRS | Glucose | 20 | 5 | 37° C. | 0 | anaerobic | | | |
| | acidophilus | | | | | | | | | | | |
| MB.I | Lactobacillus | S-XIII | MRS | Glucose | 20 | 5 | 37° C. | 0 | anaerobic | | | |
| | acidophilus | | | | | | | | | | | |
| | | | | | Infusion | | | Biomass | | Incubation | | |
| | Bacterium | | Yeast | | Stevia | | | Suspension [μl] | | Temp | | |
| Sample | Name | Strain | Name | Strain | (g/L) | sugar [g/L] | | Bacterium | Yeast | [° C.] | rpm | Gas |
| MB1 | Lactobacillus | S-IV | Meyerozyma | S-III | 60 | Sucrose | 30 | 3000 | 500 | 37 | 80 | anaerobic |
| | acidophilus | | guilliermondii | | | | | | | | | |
| MB2 | Lactobacillus | S-IV | Meyerozyma | S-IX | 60 | Sucrose | 30 | 3000 | 500 | 37 | 80 | anaerobic |
| | acidophilus | | guilliermondii | | | | | | | | | |
| MB3 | Lactobacillus | S-IV | Meyerozyma | S-X | 60 | Sucrose | 30 | 3000 | 500 | 37 | 80 | anaerobic |
| | acidophilus | | guilliermondii | | | | | | | | | |
| MB4 | Lactobacillus | S-XI | Meyerozyma | S-III | 60 | Sucrose | 30 | 3000 | 500 | 37 | 80 | anaerobic |
| | acidophilus | | guilliermondii | | | | | | | | | |
| MB5 | Lactobacillus | S-XI | Meyerozyma | S-IX | 60 | Sucrose | 30 | 3000 | 500 | 37 | 80 | anaerobic |
| | acidophilus | | guilliermondii | | | | | | | | | |
| MB6 | Lactobacillus | S-XI | Meyerozyma | S-X | 60 | Sucrose | 30 | 3000 | 500 | 37 | 80 | anaerobic |
| | acidophilus | | guilliermondii | | | | | | | | | |
| MB7 | Lactobacillus | S-XII | Meyerozyma | S-III | 60 | Sucrose | 30 | 3000 | 500 | 37 | 80 | anaerobic |
| | acidophilus | | guilliermondii | | | | | | | | | |
| MB8 | Lactobacillus | S XII | Meyerozyma | S-IX | 60 | Sucrose | 30 | 3000 | 500 | 37 | 80 | anaerobic |
| | acidophilus | | guilliermondii | | | | | | | | | |
| MB9 | Lactobacillus | S-XII | Meyerozyma | S-X | 60 | Sucrose | 30 | 3000 | 500 | 37 | 80 | anaerobic |
| | acidophilus | | guilliermondii | | | | | | | | | |
| MB10 | Lactobacillus | S-XIII | Meyerozyma | S-III | 60 | Sucrose | 30 | 3000 | 500 | 37 | 80 | anaerobic |
| | acidophilus | | guilliermondii | | | | | | | | | |
| MB11 | Lactobacillus | S-XIII | Meyerozyma | S-IX | 60 | Sucrose | 30 | 3000 | 500 | 37 | 80 | anaerobic |
| | acidophilus | | guilliermondii | | | | | | | | | |
| MB12 | Lactobacillus | S-XIII | Meyerozyma | S-X | 60 | Sucrose | 30 | 3000 | 500 | 37 | 80 | anaerobic |
| | acidophilus | | guilliermondii | | | | | | | | | |

Analytical Results:

HPLC method I: Sugar, acid analytics
Column: Rezex™ ROA-Organic Acid H+(8%), 300×4.6 mm
Solvent A: 0.1% (v/v) trifluoroacetic acid (TFA)
Flow: isocratic
Total runtime: 30 min
Detection by DAD (210 nm) and RID
Samples are diluted 2- or 5-fold (depending on expected metabolite concentrations) with appropriate volumes of 2% (v/v) and water to a final concentration of 1% (v/v) TFA and filtered (hPTFE 0.22 μm) prior to injection
Injection volume: 10 μl
Target analytes: acetic acid, formic acid, fructose, glucose, glycerol, lactic acid and succinic acid

| | | | Succinate | Lactate | Glycerol | Acetate | | | |
|---|---|---|---|---|---|---|---|---|---|
| Sample # | Glucose [mM] | Fructose [mM] | [mM] | [mM] | [mM] | [mM] | pH | Brix | OD |
| MB1 | 34.92 | 41.36 | 1.23 | 91.21 | 0.00 | 3.98 | 3.36 | 6.6 | 0.34 |
| MB2 | 31.94 | 27.43 | 1.31 | 89.41 | 0.00 | 5.34 | 3.28 | 6.4 | 0.46 |
| MB3 | 34.08 | 33.55 | 1.24 | 90.25 | 0.00 | 4.72 | 3.3 | 6.6 | 0.34 |
| MB4 | 54.55 | 32.49 | 1.52 | 77.43 | 0.00 | 4.33 | 3.39 | 6.7 | 0.32 |
| MB5 | 34.07 | 28.12 | 1.59 | 85.55 | 0.00 | 4.83 | 3.35 | 6.6 | 0.42 |
| MB6 | 43.89 | 28.05 | 1.47 | 76.11 | 0.00 | 5.08 | 3.39 | 6.7 | 0.36 |
| MB7 | 28.42 | 44.78 | 1.33 | 100.79 | 0.00 | 4.59 | 3.23 | 6.7 | 0.46 |
| MB8 | 32.77 | 30.00 | 1.33 | 100.19 | 0.00 | 4.91 | 3.23 | 6.6 | 0.52 |
| MB9 | 28.62 | 41.03 | 1.24 | 99.96 | 0.00 | 4.35 | 3.18 | 6.6 | 0.46 |
| MB10 | 32.30 | 25.93 | 0.71 | 112.82 | 0.00 | 4.07 | 3.21 | 6.3 | 0.34 |
| MB11 | 32.41 | 29.01 | 0.94 | 88.71 | 0.00 | 4.38 | 3.28 | 6.4 | 0.46 |
| MB12 | 34.12 | 29.31 | 0.89 | 110.02 | 0.00 | 3.67 | 3.22 | 6.7 | 0.34 |

HPLC method II: Qualitative Steviolglycoside analytics
Column: Kinetex C18 2.6 μm 150*2.1 mm
Solvent A: 0.1% formic acid
Solvent B: AcN+0.1% formic acid
Flow: binary gradient 0.2 mlimin starting with 20%B
Total runtime: 54 min
MS detection in negative mode 300-1300 m/z
Samples are diluted 1:10 in mobile phase (80%A/20%B) and filtrated (hPTFE 0.22 μm) prior to injection
Injection volume: 10 μl
Analyte target: Reb E, D, M, I, A, F, C, B as well as Stevioside, Dulcoside A, Rubusoside, Steviobioside

| | | | | 1 | | | 2 | | |
|---|---|---|---|---|---|---|---|---|---|
| MB | RT | | CTL | | | delta | | | delta |
| Analyte | [min] | Chromatogram | Area | Area | delta Area | Area % | Area | delta Area | Area % |
| RebE | 14.3 | EIC 965.77 -All MS | 21133166 | 33585000 | 12451834 | 58.92 | 23213272 | 2080106 | 9,84 |
| RebD | 15.9 | EIC 1127.91 -All MS | 178411712 | 271214240 | 92802528 | 52.02 | 192609712 | 14198000 | 7,96 |
| RebM | 17.8 | EIC 1290.01 -All MS | 95807944 | 169304016 | 73496072 | 76.71 | 107263096 | 11455152 | 11,96 |
| Reb O | 17.8 | EIC 1435.93 -All MS | 29082594 | 49154060 | 20071466 | 69.02 | 32391920 | 3309326 | 11,38 |
| Reb N | 18.8 | EIC 1273.93 -All MS | 100302256 | 188920432 | 88618176 | 88.35 | 117913872 | 17611616 | 17,56 |
| RebI | 26.2 | EIC 1127.91 -All MS | 42886856 | 80281056 | 37394200 | 87.19 | 49926668 | 7039812 | 16,41 |
| RebA | 26.9 | EIC 965.77 -All MS | 1239538560 | 437555648 | −801982912 | −64.70 | 1221443072 | −18095488 | -1,46 |
| Stevioside | 27.3 | EIC 849.63 -All MS | 396731136 | 1334060160 | 937329024 | 236.26 | 401947136 | 5216000 | 1,31 |
| RebF | 29.4 | EIC 935.72 -All MS | 206843424 | 374628608 | 167785184 | 81.12 | 233754576 | 26911152 | 13,01 |
| RebC | 30.5 | EIC 949.75 -All MS | 965740864 | 1263732736 | 297991872 | 30.86 | 978151680 | 12410816 | 1,29 |
| Dulcoside A | 31 | EIC 833.62 -All MS | 14314568 | 23595510 | 9280942 | 64.84 | 15326281 | 1011713 | 7,07 |
| Rubusoside | 32.1 | EIC 687.51 -All MS | 72437704 | 103103432 | 30665728 | 42.33 | 58343752 | −14093952 | -19,46 |
| RebB | 35.9 | EIC 803.65 -All MS | 222909728 | 399515072 | 176605344 | 79.23 | 270265056 | 47355328 | 21,24 |
| Steviolbioside | 36.2 | EIC 641.49 -All MS | 49181772 | 78931568 | 29749796 | 60.49 | 56349100 | 7167328 | 14,57 |

| | | | 3 | | | 4 | | |
|---|---|---|---|---|---|---|---|---|
| MB | RT | | | | delta | | | delta |
| Analyte | [min] | Chromatogram | Area | delta Area | Area % | Area | delta Area | Area % |
| RebE | 14.3 | EIC 965.77 -All MS | 31472340 | 10339174 | 48.92 | 22759620 | 1626454 | 7.70 |
| RebD | 15.9 | EIC 1127.91 -All MS | 257548000 | 79136288 | 44.36 | 188175568 | 9763856 | 5.47 |
| RebM | 17.8 | EIC 1290.01 -All MS | 147584624 | 51776680 | 54.04 | 116926720 | 21118776 | 22.04 |
| Reb O | 17.8 | EIC 1435.93 -All MS | 43392104 | 14309510 | 49.20 | 33291294 | 4208700 | 14.47 |
| Reb N | 18.8 | EIC 1273.93 -All MS | 163620384 | 63318128 | 63.13 | 126244232 | 25941976 | 25.86 |
| RebI | 26.2 | EIC 1127.91 -All MS | loniTib | 27890880 | 65.03 | 52700416 | 9813560 | 22.88 |
| RebA | 26.9 | EIC 965.77 -All MS | 1274696576 | 35158016 | 2.84 | 1191936128 | −47602432 | −3.84 |
| Stevioside | 27.3 | EIC 849.63 -All MS | 432610592 | 35879456 | 9.04 | 396234016 | −497120 | −0.13 |
| RebF | 29.4 | EIC 935.72 -All MS | 324349312 | 117505888 | 56.81 | 251034768 | 44191344 | 21.36 |
| RebC | 30.5 | EIC 949.75 -All MS | 1168051072 | 202310208 | 20.95 | 1034571648 | 68830784 | 7.13 |
| Dulcoside A | 31 | EIC 833.62 -All MS | 21982024 | 7667456 | 53.56 | 17433616 | 3119048 | 21.79 |
| Rubusoside | 32.1 | EIC 687.51 -All MS | 73725616 | 1287912 | 1.78 | 69372344 | −3065360 | −4.23 |
| RebB | 35.9 | EIC 803.65 -All MS | 355258848 | 132349120 | 59.37 | 286858656 | 63948928 | 28.69 |
| Steviolbioside | 36.2 | EIC 641.49 -All MS | 77000216 | 27818444 | 56.56 | 58756564 | 9574792 | 19.47 |
| | | | 5 | | | 6 | | |
| MB | RT | | | | delta | | | delta |
| Analyte | [min] | Chromatogram | Area | delta Area | Area % | Area | delta Area | Area % |
| RebE | 14.3 | EIC 965.77 -All MS | 29691366 | 8558200 | 40.50 | 20023090 | −1110076 | −5.25 |
| RebD | 15.9 | EIC 1127.91 -All MS | 246800176 | 68388464 | 38.33 | 170226688 | −8185024 | −4.59 |
| RebM | 17.8 | EIC 1290.01 -All MS | 136609232 | 40801288 | 42.59 | 92894928 | −2913016 | −3.04 |
| Reb O | 17.8 | EIC 1435.93 -All MS | 41093028 | 12010434 | 41.30 | 28715204 | −367390 | −1.26 |
| Reb N | 18.8 | EIC 1273.93 -All MS | 153323232 | 53020976 | 52.86 | 99715488 | −586768 | −0.58 |
| RebI | 26.2 | EIC 1127.91 -All MS | 62650256 | 19763400 | 46.08 | 41810476 | −1076380 | −2.51 |
| RebA | 26.9 | EIC 965.77 -All MS | 1248640000 | 9101440 | 0.73 | 1189953536 | −49585024 | −4.00 |
| Stevioside | 27.3 | EIC 849.63 -All MS | 412150688 | 15419552 | 3.89 | 382441440 | −14289696 | −3.60 |
| RebF | 29.4 | EIC 935.72 -All MS | 303273024 | 96429600 | 46.62 | 204290000 | −2553424 | −1.23 |
| RebC | 30.5 | EIC 949.75 -All MS | 1110822784 | 145081920 | 15.02 | 878784064 | −86956800 | −9.00 |
| Dulcoside A | 31 | EIC 833.62 -All MS | 18564422 | 4249854 | 29.69 | 13486525 | −828043 | −5.78 |
| Rubusoside | 32.1 | EIC 687.51 -All MS | 69059248 | −3378456 | −4.66 | 43380476 | −29057228 | −40.11 |
| RebB | 35.9 | EIC 803.65 -All MS | 339326944 | 116417216 | 52.23 | 233006368 | 10096640 | 4.53 |
| Steviolbioside | 36.2 | EIC 641.49 -All MS | 72466744 | 23284972 | 47.34 | 48258656 | −923116 | −1.88 |
| | | | 7 | | | 8 | | |
| MB | RT | | | | delta | | | delta |
| Analyte | [min] | Chromatogram | Area | delta Area | Area % | Area | delta Area | Area % |
| RebE | 14.3 | EIC 965.77 -All MS | 26575318 | 5442152 | 25.75 | 20169920 | −963246 | −4.56 |
| RebD | 15.9 | EIC 1127.91 -All MS | 232754208 | 54342496 | 30.46 | 183348816 | 4937104 | 2.77 |
| RebM | 17.8 | EIC 1290.01 -All MS | 141978624 | 46170680 | 48.19 | 99411608 | 3603664 | 3.76 |
| Reb O | 17.8 | EIC 1435.93 -All MS | 41670308 | 12587714 | 43.28 | 30435010 | 1352416 | 4.65 |
| Reb N | 18.8 | EIC 1273.93 -All MS | 156858816 | 56556560 | 56.39 | 108172936 | 7870680 | 7.85 |
| RebI | 26.2 | EIC 1127.91 -All MS | 65344568 | 22457712 | 52.37 | 44067180 | 1180324 | 2.75 |
| RebA | 26.9 | EIC 965.77 -All MS | 1281823360 | 42284800 | 3.41 | 1142243328 | −97295232 | −7.85 |
| Stevioside | 27.3 | EIC 849.63 -All MS | 430535840 | 33804704 | 8.52 | 378334688 | −18396448 | −4.64 |
| RebF | 29.4 | EIC 935.72 -All MS | 306966368 | 100122944 | 48.41 | 213909056 | 7065632 | 3.42 |
| RebC | 30.5 | EIC 949.75 -All MS | 1158836096 | 193095232 | 19.99 | 910271040 | −55469824 | −5.74 |
| Dulcoside A | 31 | EIC 833.62 -All MS | 18774058 | 4459490 | 31.15 | 14881779 | 567211 | 3.96 |
| Rubusoside | 32.1 | EIC 687.51 -All MS | 90684848 | 18247144 | 25.19 | 48337536 | −24100168 | −33.27 |
| RebB | 35.9 | EIC 803.65 -All MS | 352158208 | 129248480 | 57.98 | 239110448 | 16200720 | 7.27 |
| Steviolbioside | 36.2 | EIC 641.49 -All MS | 70024360 | 20842588 | 42.38 | 51668700 | 2486928 | 5.06 |
| | | | 9 | | | 10 | | |
| MB | RT | | | | delta | | | delta |
| Analyte | [min] | Chromatogram | Area | delta Area | Area % | Area | delta Area | Area % |
| RebE | 14.3 | EIC 965.77 -All MS | 27044714 | 5911548 | 27.97 | 25991984 | 4858818 | 22.99 |
| RebD | 15.9 | EIC 1127.91 -All MS | 227528000 | 49116288 | 27.53 | 229175552 | 50763840 | 28.45 |
| RebM | 17.8 | EIC 1290.01 -All MS | 135143184 | 39335240 | 41.06 | 138206864 | 42398920 | 44.25 |
| Reb O | 17.8 | EIC 1435.93 -All MS | 38367936 | 9285342 | 31.93 | 42423200 | 13340606 | 45.87 |
| Reb N | 18.8 | EIC 1273.93 -All MS | 140139440 | 39837184 | 39.72 | 150341264 | 50039008 | 49.89 |
| RebI | 26.2 | EIC 1127.91 -All MS | 57723868 | 14837012 | 34.60 | 64493716 | 21606860 | 50.38 |
| RebA | 26.9 | EIC 965.77 -All MS | 1215893760 | −23644800 | −1.91 | 1235655680 | −3882880 | −0.31 |
| Stevioside | 27.3 | EIC 849.63 -All MS | 403002592 | 6271456 | 1.58 | 414062240 | 17331104 | 4.37 |
| RebF | 29.4 | EIC 935.72 -All MS | 278342688 | 71499264 | 34.57 | 293842208 | 86998784 | 42.06 |
| RebC | 30.5 | EIC 949.75 -All MS | 1073526656 | 107785792 | 11.16 | 1125065088 | 159324224 | 16.50 |
| Dulcoside A | 31 | EIC 833.62 -All MS | 19572422 | 5257854 | 36.73 | 19836306 | 5521738 | 38.57 |
| Rubusoside | 32.1 | EIC 687.51 -All MS | 64959744 | −7477960 | −10.32 | 76920296 | 4482592 | 6.19 |
| RebB | 35.9 | EIC 803.65 -All MS | 316615904 | 93706176 | 42.04 | 323027328 | 100117600 | 44.91 |
| Steviolbioside | 36.2 | EIC 641.49 -All MS | 66637128 | 17455356 | 35.49 | 66778728 | 17596956 | 35.78 |
| | | | 11 | | | 12 | | |
| MB | RT | | | | delta | | | delta |
| Analyte | [min] | Chromatogram | Area | delta Area | Area % | Area | delta Area | Area % |
| RebE | 14.3 | EIC 965.77 -All MS | 23156738 | 2023572 | 9.58 | 20479096 | −654070 | −3.09 |
| RebD | 15.9 | EIC 1127.91 -All MS | 207243104 | 28831392 | 16.16 | 174267728 | −4143984 | −2.32 |
| RebM | 17.8 | EIC 1290.01 -All MS | 119581232 | 23773288 | 24.81 | 99081224 | 3273280 | 3.42 |
| Reb O | 17.8 | EIC 1435.93 -All MS | 35129796 | 6047202 | 20.79 | 29828870 | 746276 | 2.57 |
| Reb N | 18.8 | EIC 1273.93 -All MS | 130991576 | 30689320 | 30.60 | 110230112 | 9927856 | 9.90 |
| RebI | 26.2 | EIC 1127.91 -All MS | 51848760 | 8961904 | 20.90 | 43290368 | 403512 | 0.94 |
| RebA | 26.9 | EIC 965.77 -All MS | 1204701952 | −34836608 | −2.81 | 1156384128 | −83154432 | −6.71 |
| Stevioside | 273 | EIC 849.63 -All MS | 414527328 | 17796192 | 4.49 | 387886144 | −8844992 | −2.23 |
| RebF | 29.4 | EIC 935.72 -All MS | 255493728 | 48650304 | 23.52 | 215662720 | 8819296 | 4.26 |
| RebC | 30.5 | EIC 949.75 -All MS | 1048821056 | 83080192 | 8.60 | 905791424 | −59949440 | −6.21 |
| Dulcoside A | 31 | EIC 833.62 -All MS | 16716718 | 2402150 | 16.78 | 14747960 | 433392 | 3.03 |
| Rubusoside | 32.1 | EIC 687.51 -All MS | 75089040 | 2651336 | 3.66 | 55947696 | −16490008 | −22.76 |
| RebB | 35.9 | EIC 803.65 -All MS | 302767328 | 79857600 | 35.83 | 238574176 | 15664448 | 7.03 |
| Steviolbioside | 36.2 | EIC 641.49 -All MS | 64069524 | 14887752 | 30.27 | 49876788 | 695016 | 1.41 |

Sensory results:

Fermented samples were applied in a beverage composition as follows:
Prepare a concentrated base of the drink that will be diluted 1:4 (1 part of base for 4 parts of water).
Add the ingredient at 10 g/L
Top up with the remaining part of water.
Apple/Cherry drink:
BASE INGREDIENTS:
Ingredients: Water, Sugar, Fruit juices from concentrates 5% (Apple, Cherry), Natural flavourings (Apple, Cherry), Acids (Citric acid, Ascorbic acid), Acidity regulator (Sodium gluconate), Colour (extracts of carrot, hibiscus), Sweetener (Steviol glycosides)

Fermented samples were tasted by a trained expert comparing samples to an internal benchmark.

| | | Description on |
|---|---|---|
| | | overall flavour |
| Sample # | Description on sweetness | impression |
| MB1 | Clean sweetness, no lingering | floral notes, fruity, clean, |
| | aftertaste, reduced | similar sweetness |
| | bitterness from Stevia | and mouthfeel, drying |
| | | aftertaste |
| MB2 | Clean sweetness, no lingering | Drying, bland, clean |
| | aftertaste, reduced | |
| | bitterness from Stevia | |
| MB3 | Clean sweetness, no lingering | Bland, clean, watery |
| | aftertaste, reduced | |
| | bitterness from Stevia | |
| MB4 | Clean sweetness, no lingering | Clean,fruity, less |
| | aftertaste, reduced | mouthfeel |
| | bitterness from Stevia | |
| MB5 | Clean sweetness, no lingering | Clean, drying, watery |
| | aftertaste, reduced | |
| | bitterness from Stevia | |
| MB6 | Clean sweetness, no lingering | Fruity, clean, similar |
| | aftertaste, reduced | mouthfeel and |
| | bitterness from Stevia | sweetness |
| MB7 | Clean sweetness, no lingering | Floral tea notes, |
| | aftertaste, reduced | similar sweetness |
| | bitterness from Stevia | and mouthfeel |
| MB8 | Clean sweetness, no lingering | Clean, fruity, similar |
| | aftertaste, reduced | sweetness and |
| | bitterness from Stevia | mouthfeel |
| MB9 | Clean sweetness, no lingering | Clean,fruity, less |
| | aftertaste, reduced | mouthfeel |
| | bitterness from Stevia | |
| MB10 | Clean sweetness, no lingering | Bland, clean |
| | aftertaste, reduced | |
| | bitterness from Stevia | |
| MB11 | Clean sweetness, no lingering | Sour, bland, clean |
| | aftertaste, reduced | |
| | bitterness from Stevia | |
| MB12 | Clean sweetness, no lingering | Bland, clean |
| | aftertaste, reduced | |
| | bitterness from Stevia | |

### Example 10

### Screening of Fermentation Microorganisms

Small samples (50 ml) of a 60 g/L stevia infusion, prepared as described herein, were supplemented with 30 g/L sugar and then inoculated with various fermentation microorganisms and incubated in 300 ml shake flasks. Microbial strains were cultivated on a small scale in shake flasks, harvested after 2 days, suspended in spent medium and subsequently served as inoculum in the infusion. Duration of fermentation step was 3 days. Harvesting was done as in example 7A.

Experimental parameters:

| | | | | | | resuspend in | | | | | | |
|---|---|---|---|---|---|---|---|---|---|---|---|---|
| Strain | | | Medium | | | spent | | | | | | |
| Culture | Species | Strain | | Sugar g/L | | medium [ml] | Temp | rpm | Gas | | | |
| | | | 30 ml Medium, | | | | | | | | | |
| | | | 300 ml shakeflask | | | | | | | | | |
| MC.A1 | Pichia | S-I | YPD | Glucose | 15 | 3 | 28° C. | 180 | aerobic | | | |
| | membranifaciens | | | | | | | | | | | |
| MC.A2 | Cyberlindnera | S-II | YPD | Glucose | 15 | 3 | 28° C. | 180 | aerobic | | | |
| | jadinii | | | | | | | | | | | |
| MC.A3 | Meyerozyma | S-III | YPD | Glucose | 15 | 3 | 28° C. | 180 | aerobic | | | |
| | guilliermondii | | | | | | | | | | | |
| | | | 150 ml Medium, | | | | | | | | | |
| | | | 300 ml shakeflask | | | | | | | | | |
| MC.B | Lactobacillus | S-IV | MRS | Glucose | 20 | 5 | 37° C. | 0 | anaerobic | | | |
| | acidophilus | | | | | | | | | | | |
| MC.C | Lactobacillus | S-V | MRS | Glucose | 20 | 5 | 37° C. | 0 | anaerobic | | | |
| | delbrueckii | | | | | | | | | | | |
| | | | 50 ml Medium, | | | | | | | | | |
| | | | 500 ml shakeflask | | | | | | | | | |
| MC.D | Lactobacillus | S-VI | MRS | Glucose | 20 | 1.5 | 37° C. | 0 | anaerobic | | | |
| | fructivorans | | | | | | | | | | | |
| MC.E | Lactobacillus | S-VI | MRS | Glucose | 20 | 1.5 | 37° C. | 0 | anaerobic | | | |
| | fructivorans | | | | | | | | | | | |
| MC.F | Lactobacillus | S-VI | MRS | Glucose | 20 | 1.5 | 37° C. | 0 | anaerobic | | | |
| | fructivorans | | | | | | | | | | | |
| MC.G | Lactobacillus | S-VI | MRS | Glucose | 20 | 1.5 | 28° C. | 180 | aerobic | | | |
| | fructivorans | | | | | | | | | | | |
| MC.H | Lactococcus | S-VIII | MRS | Glucose | 20 | 1.5 | 28° C. | 180 | aerobic | | | |
| | raffinolactis | | | | | | | | | | | |
| MA.D3 | | | MRS | Glucose | 20 | 1.5 | 28° C. | 180 | aerobic | | | |
| MA.E1 | | | MRS | Glucose | 20 | 2 | 28° C. | 180 | aerobic | | | |
| MA.E2 | | | MRS | Glucose | 20 | 2 | 28° C. | 180 | aerobic | | | |
| MA.F1 | | | MRS | Glucose | 20 | 2 | 28° C. | 180 | aerobic | | | |
| MA.F2 | | | MRS | Glucose | 20 | 2 | 28° C. | 180 | aerobic | | | |
| | | | | | Infusion | | | Biomass | | Incubation | | |
| | Bacterium | | Yeast | | Stevia | | | Suspension [μl] | | Temp | | |
| Sample | Name | Strain | Name | Strain | (g/L) | sugar [g/L] | | Bacterium | Yeast | [° C.] | rpm | Gas |
| MC1 | Lactobacillus | S-IV | | | 60 | Sucrose | 30 | 2000 | | 37 | 80 | anaerobic |
| | acidophilus | | | | | | | | | | | |
| MC2 | Lactobacillus | S-V | | | 60 | Sucrose | 30 | 2000 | | 37 | 80 | anaerobic |
| | delbrueckii | | | | | | | | | | | |
| MC3 | Lactobacillus | S-VI | | | 60 | Sucrose | 30 | 2000 | | 28 | 80 | aerobic |
| | fructivorans | | | | | | | | | | | |
| MC4 | Lactobacillus | S-IV | Pichia | S-I | 60 | Sucrose | 30 | 1000 | 200 | 37 | 80 | anaerobic |
| | acidophilus | | membranifaciens | | | | | | | | | |
| MC5 | Lactobacillus | S-V | Pichia | S-I | 60 | Sucrose | 30 | 1000 | 200 | 37 | 80 | anaerobic |
| | delbrueckii | | membranifaciens | | | | | | | | | |
| MC6 | Lactobacillus | S-VI | Pichia | S-I | 60 | Sucrose | 30 | 1000 | 200 | 28 | 80 | aerobic |
| | fructivorans | | membranifaciens | | | | | | | | | |
| MC7 | Lactobacillus | S-IV | Cyberlindnera | S-II | 60 | Sucrose | 30 | 1000 | 200 | 37 | 80 | anaerobic |
| | acidophilus | | jadinii | | | | | | | | | |
| MC8 | Lactobacillus | S-V | Cyberlindnera | S-II | 60 | Sucrose | 30 | 1000 | 200 | 37 | 80 | anaerobic |
| | delbrueckii | | jadinii | | | | | | | | | |
| MC9 | Lactobacillus | S-VI | Cyberlindnera | S-II | 60 | Sucrose | 30 | 1000 | 200 | 28 | 80 | aerobic |
| | fructivorans | | jadinii | | | | | | | | | |
| MC10 | Lactobacillus | S-V | Meyerozyma | S-III | 60 | Sucrose | 30 | 1000 | 200 | 37 | 80 | anaerobic |
| | delbrueckii | | guilliermondii | | | | | | | | | |
| MC11 | Lactobacillus | S-VI | Meyerozyma | S-III | 60 | Sucrose | 30 | 1000 | 200 | 28 | 80 | aerobic |
| | fructivorans | | guilliermondii | | | | | | | | | |
| MC12 | Lactococcus | S-VIII | Meyerozyma | S-III | 60 | Sucrose | 30 | 1000 | 200 | 37 | 80 | anaerobic |
| | raffinolactis | | guilliermondii | | | | | | | | | |

Analytical results:

HPLC method I: Sugar, acid analytics
Column: Rezex™ ROA-Organic Acid H+(8%), 300×4.6 mm
Solvent A: 0,1% (v/v) trifluoroacetic acid (TFA)
Flow: isocratic
Total runtime: 30 min
Detection by DAD (210 nm) and RID
Samples are diluted 2- or 5-fold (depending on expected metabolite concentrations) with appropriate volumes of 2% (v/v) and water to a final concentration of 1% (v/v) TFA and filtered (hPTFE 0.22 μm) prior to injection
Injection volume: 10 μl
Target analytes: acetic acid, formic acid, fructose, glucose, glycerol, lactic acid and succinic acid

| | Glucose | Fructose | Succinate | Lactate | Glycerol | Acetate | | | |
|---|---|---|---|---|---|---|---|---|---|
| Sample # | [mM] | [mM] | [mM] | [mM] | [mM] | [mM] | PH | Brix | OD |
| MC1 | 95.05 | 86.73 | 1.38 | 62.31 | 0.00 | 4.30 | 3.25 | 6 | 0.19 |
| MC2 | 79.24 | 83.88 | 1.09 | 58.99 | 0.00 | 4.03 | 3.23 | 5.8 | 0.21 |
| MC3 | 40.79 | 33.80 | 0.00 | 57.45 | 2.43 | 47.43 | 3.11 | 5.2 | 0.25 |
| MC4 | 89.89 | 87.60 | 1.61 | 52.46 | 0.00 | 0.00 | 3.22 | 5.8 | 0.26 |
| MC5 | 87.19 | 94.16 | 1.35 | 46.59 | 0.00 | 0.00 | 3.28 | 5.8 | 0.27 |
| MC6 | 33.25 | 53.62 | 0.00 | 47.69 | 1.95 | 41.23 | 3.11 | 4.8 | 0.28 |
| MC7 | 0.16 | 0.71 | 4.50 | 20.52 | 17.57 | 4.59 | 3.39 | 3.2 | 0.23 |
| MC8 | 0.24 | 0.33 | 4.23 | 24.31 | 17.35 | 7.20 | 3.42 | 3.2 | 0.27 |
| MC9 | 0.83 | 0.00 | 1.47 | 22.42 | 10.48 | 26.09 | 3.4 | 3.5 | 0.3 |
| MC10 | 95.74 | 105.25 | 1.03 | 41.25 | 0.00 | 0.00 | 3.35 | 5.9 | 0.31 |
| MC11 | 0.49 | 0.00 | 0.00 | 56.27 | 6.29 | 69.49 | 3.02 | 4.4 | 0.25 |
| MC12 | 96.07 | 102.37 | 0.67 | 6.44 | 0.24 | 0.00 | 4.43 | 5.8 | 0.23 |

Sensory Results (Trained Expert):

Fermented samples were applied in a beverage composition as follows:
Prepare a concentrated base of the drink that will be diluted 1:4 (1 part of base for 4 parts of water).
Add the ingredient at 10 g/L
Top up with the remaining part of water.
Apple/Cherry drink:
BASE INGREDIENTS:
Ingredients: Water, Sugar, Fruit juices from concentrates 5% (Apple, Cherry), Natural flavourings (Apple, Cherry), Acids (Citric acid, Ascorbic acid), Acidity regulator (Sodium gluconate), Colour (extracts of carrot, hibiscus), Sweetener (Steviol glycosides)

Fermented samples were tasted by a trained expert comparing samples against an internal benchmark

| Sample # | Description on sweetness |
|---|---|
| MC1 | Clean sweetness, no lingering aftertaste, |
| | reduced bitterness from Stevia |
| MC2 | Clean sweetness, no lingering aftertaste, |
| | reduced bitterness from Stevia |
| MC3 | Clean sweetness, no lingering aftertaste, |
| | reduced bitterness from Stevia |
| MC4 | Clean sweetness, no lingering aftertaste, |
| | reduced bitterness from Stevia |
| MC5 | Clean sweetness, no lingering aftertaste, |
| | reduced bitterness from Stevia |
| MC6 | Clean sweetness, no lingering aftertaste, |
| | reduced bitterness from Stevia |
| MC7 | Clean sweetness, no lingering aftertaste, |
| | reduced bitterness from Stevia |
| MC8 | Clean sweetness, no lingering aftertaste, |
| | reduced bitterness from Stevia |
| MC9 | Clean sweetness, no lingering aftertaste, |
| | reduced bitterness from Stevia |
| MC10 | Clean sweetness, no lingering aftertaste, |
| | reduced bitterness from Stevia |
| MC11 | Clean sweetness, no lingering aftertaste, |
| | reduced bitterness from Stevia |
| MC12 | Clean sweetness, no lingering aftertaste, |
| | reduced bitterness from Stevia |

### Example 11

To explore the utility of the ingredient of the invention as a sweetening ingredient for food (e.g. shortbread) the following trial was carried out.

The objective is to understand consumer perception and the main differences between four shortbreads:

Full sugar (shortbread A)

Half sugar +fermented stevia infusion according to the invention (shortbread B)

Half sugar +unfermented stevia infusion (shortbread C)

Half sugar +Reb A (shortbread D)

Ingredients:

1 kg of each shortbread batch was made according to the recipes below (amounts in wt %):

| | A | B | C | D |
|---|---|---|---|---|
| Butter | 45 | 45 | 45 | 45 |
| Flour | 30 | 30 | 30 | 30 |
| Sugar | 20 | 10 | 10 | 10 |
| Polydextrose | — | 10 | 10 | 10 |
| Fermented stevia infusion | — | 5.7 | — | — |
| Unermented stevia infusion | — | — | 5.7 | — |
| Reb A | — | — | — | 0.045 |
| Water | 5 | — | — | 5 |

Erythritol formulas were used to determine equivalent amounts of each sweetener to achieve the same theoretical sweetness.

All doughs were rolled out to approximately 1 cm in thickness and 5 cm in diameter and cooked in an oven at 160° C. for 25 minutes.

Cookies were cooled and packaged up to be sent to participants for tasting.

Method:
A total of 11 people evaluated the samples in an online test.
Participants assessed samples following a questionnaire about their personal taste and perception, being a subjective methodology.
Data was captured on an online survey application, and then analysed with XLSTAT.
For each question, statistical analysis of data through ANOVA and multiple range test (LSD) distributed products into different groups (identified with alphabetic letters) according to the sequential order and the significant differences among samples means.
All differences referred to are significant at 95% confidence level.

No significant difference was found between the samples for overall liking and taste liking.

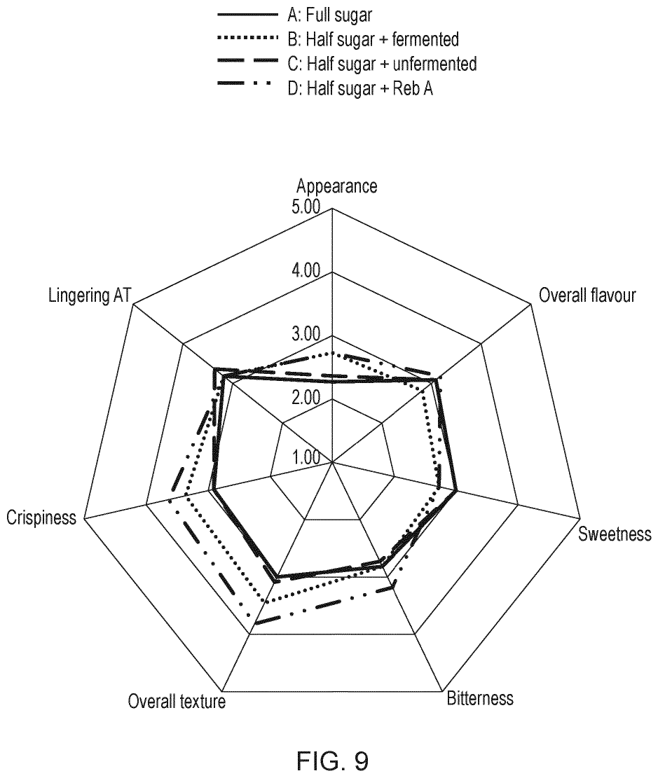

Exemplary sensory results (JAR questions) are shown in FIG. 9.

Appearance: B and D are very similar. The other samples are lighter in colour

Overall flavor: B is the least intense

Sweetness: B and A are perceived similarly in sweetness (Just-About-Right).

Bitterness: D is the most bitter.

Overall texture: D is the driest and A is Just-About-Right.

Crispiness: D is the crispiest. A and C are perceived similarly.

Lingering aftertaste (AT): C is slightly more lingering than the other samples.

1. An ingredient based on a fermented infusion of stevia, wherein the infusion has been fermented using a combination of at least two different microorganisms.

2. An ingredient according to claim 1, wherein the infusion has been fermented using a combination of at least one yeast and at least one bacteria.

3. An ingredient according to claim 2, wherein the yeast is selected from Saccharomyces cerevisiae, Kluyveromyces lactis, Kluyveromyces marxianus, Zygosaccharomyces rouxii, Pichia membranifaciens, Cyberlindnera jadinii, and Meyerozyma guilliermondii.

4. An ingredient according to claim 2, wherein the yeast is Meyerozyma guilliermondii.

5. An ingredient according to claim 2, wherein at least one bacteria is a lactic-acid producing bacteria, preferably of the Lactobacillus genus, more preferably selected from L. acidophilus, L. fructivorans, L. gasseri, L. jensenii, L. delbrueckii, L. delbrueckii subsp. bulgaricus, L. amylovorus, L. crispatus, and L. helveticus.

6. An ingredient according to claim 2, wherein at least one bacteria is Lactobacillus acidophilus.

7. An ingredient according to claim 1, wherein fermentation with each microorganism takes place sequentially.

8. An ingredient according to claim 1, wherein fermentation with each microorganism takes place simultaneously.

9. An ingredient comprising a fermented infusion of stevia, wherein the infusion is fermented by a microorganism selected from yeast and bacteria, or a combination thereof, wherein the fermented infusion is prepared by contacting uncured stevia leaves with water to produce an infusion, then adding a fermentation microorganism to said infusion.

10. An ingredient according to claim 1, wherein the sweetening ingredient has a taste and sensory profile which is modified and/or improved as a result of the fermentation, when compared to an unfermented stevia infusion.

11. An ingredient according to claim 9, wherein the infusion is fermented using yeast, for example a yeast selected from Saccharomyces cerevisiae, Kluyveromyces lactis, Kluyveromyces marxianus, Zygosaccharomyces rouxii, Pichia membranifaciens, Cyberlindnera jadinii, and Meyerozyma guilliermondii.

12. An ingredient according to claim 9, wherein the infusion is fermented using bacteria, for example a lactic acid producing bacterium.

13. An ingredient according to claim 9, wherein the infusion is fermented using more than one microorganism, wherein fermentation with each microorganism takes place either sequentially or simultaneously.

14. An ingredient according to claim 13, wherein the infusion is fermented using a combination of one or more yeasts with one or more bacteria.

15. An ingredient according to claim 1, wherein the microorganism or combination of microorganisms is selected to provide a pre-determined sensory and/or taste profile in the fermented infusion.

16. An ingredient according to claim 1, wherein the fermented infusion is prepared by contacting stevia with water to produce an infusion, then adding the microorganism directly to said infusion to perform the fermentation.

17. An ingredient according to claim 1 wherein the infusion is produced by heating, preferably at a temperature below about 90° C., more preferably at a temperature below 85° C., more preferably at a temperature of 50-70° C.

18. An ingredient according to claim 1, wherein the stevia comprises plant material from Stevia rebaudiana, Stevia phlebophylla, Rubus chingii, Rubus suavissimus or Siraitia grosvenorii.

19. An ingredient according to claim 1, wherein the stevia comprises plant material from Stevia rebaudiana.

20. An ingredient according to claim 1 wherein the fermented infusion has a modified steviol glycoside composition compared to the unfermented infusion.

21. (canceled)

22. (canceled)

23. An ingredient comprising an aqueous solution of steviol glycosides, having:
a pH from about 3.1 to about 3.9;
an OD₆₀₀ from about 0.15 to 0.8;
a lactate content from about 0 to about 10 g/L; and
an acetate content from about 0 to about 2.5 g/L.

24. An ingredient according to claim 23, wherein the ingredient comprises at least 100 μm steviol glycosides in solution.

25. (canceled)

26. An ingredient according to claim 23 wherein the ingredient is obtainable by or obtained by a process involving fermention of a stevia infusion.

27. A process for preparing an ingredient comprising a fermented infusion of stevia, said process comprising the steps of:
(a) contacting stevia with water and optionally heating to produce an infusion;
(b) optionally adding carbohydrate or a carbohydrate source to the infusion;
(c) optionally filtering the infusion to remove remaining stevia;
(d) adding a fermentation microorganism to the infusion;
(e) fermenting the infusion under conditions suitable to the microorganism, to produce a fermented infusion.

28. A process according to claim 27, wherein step (a) comprises heating at a temperature of 40-90° C.

29. A process according to claim 27, wherein in step (a) the duration of the infusion step is from 15 to 45 minutes.

30. A process according to claim 27, wherein the carbohydrate added in step (b) is sugar and the total amount of sugar at the start of the fermentation step is from 30-50 g/L (3-5 Bx).

31. A process according to claim 27, wherein the microorganism used for the fermentation is selected from yeast or bacteria, or a combination thereof.

32. A process according to claim 31, wherein the microorganism used for the fermentation step comprises a yeast selected from Saccharomyces cerevisiae, Kluyveromyces lactis, Kluyveromyces marxianus, Zygosaccharomyces rouxii, Pichia membranifaciens, Cyberlindnera jadinii, and Meyerozyma guilliermondii.

33. A process according to claim 31, wherein the microorganism used for the fermentation step comprises bacteria.

34. A process according to claim 27, wherein the microorganism used for the fermentation is selected to provide a pre-determined sensory and/or taste profile in the fermented infusion.

35. (canceled)

36. (canceled)

37. (canceled)

38. Use of a sweetening ingredient according to claim 1, in the production of a food or beverage product.

39. A food or beverage product comprising a sweetening ingredient according to claim 1.

40. (canceled)

41. (canceled)

42. (canceled)
